(19) United States
(12) Patent Application Publication
(10) Pub. No.: US 2020/0009204 A1
(43) Pub. Date: Jan. 9, 2020

(54) USE OF ONCOLYTIC VIRUSES, ALONE OR IN COMBINATION WITH A CHECKPOINT INHIBITOR, FOR THE TREATMENT OF CANCER

(71) Applicant: AMGEN INC., Thousand Oaks, CA (US)

(72) Inventors: Pedro J. BELTRAN, Orinda, CA (US); Courtney BEERS, San Francisco, CA (US); Keegan COOKE, Ventura, CA (US)

(21) Appl. No.: 16/493,376

(22) PCT Filed: Mar. 14, 2018
(86) PCT No.: PCT/US2018/022442

§ 371 Date: Sep. 12, 2019

Related U.S. Application Data
(60) Provisional application No. 62/471,875, filed on Mar. 15, 2017.

(51) Int. Cl. A61K 35/763; C07K 16/28; A61P 35/00

(52) U.S. Cl. CPC A61K 35/763; A61P 35/00; C07K 16/2827; C07K 16/2818

(57) ABSTRACT

The present invention relates to the use of oncolytic viruses (e.g., talimogene laherparepvec), either alone or in combination with immune checkpoint inhibitors (e.g., anti-CTLA-4, anti-PD-1, and anti-PD-L1 compounds such an antibodies) for the treatment of various types of cancer. In addition, the present invention relates to compositions and kits relating to such uses of oncolytic viruses, either alone or in combination with immune checkpoint inhibitors.

### CROSS REFERENCE TO RELATED APPLICATIONS

This application claims the benefit under 35 U.S.C. 119(e) of U.S. provisional patent application No. 62/471,875, filed Mar. 15, 2017, which is incorporated herein by reference.

### BACKGROUND OF THE INVENTION

In light of advances in cancer therapy over the past few decades, the rate of death from cancer continues to decline among both men and women for the most common types of cancer such as lung cancer, colon cancer, breast cancer, and prostate cancer. This improvement in survival likely stems from progress in diagnosing certain cancers at an earlier stage, improvements in treatment, and the results of public health initiatives encouraging preventative measures and screening.

Still, cancer remains a major public health problem with more than 1.6 million people diagnosed each year. In addition, cancer diagnoses have profound effects on patients as well as their families and friends. Indeed, cancer remains the second most common cause of death in the United States (exceeded only by heart disease) and accounts for nearly one in every four deaths. See, progressreport.cancer.gov/introduction; accessed Mar. 8, 2017.

The desired goal of cancer therapy is to preferentially kill cancer cells without having a deleterious effect on normal cells. Several methods have been used in an attempt to reach this goal, including surgery, radiation therapy, chemotherapy, and therapy with oncolytic viruses.

Local treatments, such as radiation therapy and surgery, offer a way of reducing the tumor mass in regions of the body that are accessible through surgical techniques or high doses of radiation therapy. The primary approach for cancer treatment, however, is chemotherapy. Yet, chemotherapeutic agents are limited in their effectiveness for treating many cancer types, including many common solid tumors. This failure is in part due to the drug resistance (whether acquired or intrinsic) of many tumor cells. A serious drawback to the use of chemotherapeutic agents is their severe side effects. These include bone marrow suppression, nausea, vomiting, hair loss, and ulcerations in the mouth.

Proposed alternative therapies include the administration of oncolytic viruses, and the use of viral vectors to deliver a transgene with anti-cancer activity. The genetic engineering of viruses for use as oncolytic agents initially focused on the use of replication-incompetent viruses in a bid to prevent virus-induced damage to non-tumor cells. A major limitation of this approach was that these replication-incompetent viruses required a helper virus to be able to integrate and/or replicate in a host cell. These viruses are limited in their effectiveness, because each replication-defective retrovirus particle can enter only a single cell and cannot productively infect others thereafter. Therefore, they cannot spread far from the producer cell, and are unable to completely penetrate many tumors in vivo. More recently, genetic engineering of oncolytic viruses has focused on the generation of “replication-conditional” viruses in an effort to avoid systemic infection while allowing the virus to spread to other tumor cells.

Currently, the only approved oncolytic virus-based drug in the U.S. and Europe is talimogene laherparepvec (IMLYGIC®). Talimogene laherparepvec is an HSV-1 derived from the clinical strain JS1 (deposited at the European collection of cell cultures (ECAAC) under accession number 01010209). In talimogene laherparepvec, the HSV-1 viral genes encoding ICP34.5 and ICP47 have been functionally deleted. Functional deletion of ICP47 leads to earlier expression of US11, a gene that promotes virus growth in tumor cells without decreasing tumor selectivity. In addition, the coding sequence for human GM-CSF has been inserted into the viral genome at the former ICP34.5 gene sites. See, Liu et al., Gene Ther., 10:292-303, 2003.

Therapeutic combinations of oncolytic viruses and checkpoint inhibitors have been explored. For example, combinations of talimogene laherparepvec and immunotherapies (e.g., ipilimumab and pembrolizumab) are currently being explored in clinical trials in melanoma (NCT01740297 and NCT02263508) and squamous cell carcinoma of the head and neck (NCT02626000). Checkpoint inhibitors, such as ipilimumab (an CTLA-4 antibody), pembrolizumab and nivolumab (anti-PD-1 antibodies), and atezolizumab (an anti-PD-L1 antibody) have demonstrated efficacy in a variety of tumor types. See, Grosso et al., Cancer Immun., 13:5 (2013); Pardoll, Nat Rev Cancer, 12:252-264 (2012); and Chen et al., Immunity, 39:1-10 (2013).

However, there remains a need to further develop effective cancer therapies with diminished side effects (e.g., compared to chemotherapy). There also remains a need to further develop effective cancer therapies which are effective against metastatic cancers. The present invention addresses these and other needs.

### SUMMARY OF THE INVENTION

In one embodiment, the present invention relates to a method of treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal (i.e., colon cancer), melanoma, squamous carcinoma (e.g., head and neck squamous carcinoma), hepatocellular carcinoma, gastric carcinoma, breast cancer (e.g., triple negative breast carcinoma), cutaneous T-cell lymphoma, or multiple myeloma by administering a therapeutically effective amount of an oncolytic virus. In some embodiments, the cancer is a metastatic cancer. In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. The herpes simplex virus 1 may also be modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

The present invention also relates to a method of treating B-cell lymphoma, colorectal cancer, melanoma, head and neck squamous carcinoma, or breast cancer (e.g., triple negative breast carcinoma), by administering: (i) a therapeutically effective amount of an oncolytic virus; and (ii) a therapeutically effective amount of a checkpoint inhibitor. In some embodiments, the cancer is a metastatic cancer. In some embodiments, the checkpoint inhibitor is a CTLA-4 blocker (e.g., an anti-CTLA-4 antibody). In particular embodiments, the anti-CTLA-4 antibody is ipilimumab. In some embodiments, the checkpoint inhibitor is a PD-L1 blocker (e.g., an anti-PD-L1 antibody). In particular embodiments, the anti-PD-L1 antibody is atezolizumab. In some embodiments, the checkpoint inhibitor is a PD-1 blocker (e.g., an anti-PD-1 antibody). In particular embodiments, the anti-PD-1 antibody is: nivolumab or pembrolizumab. In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. The herpes simplex virus 1 may also be modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

In a specific embodiment, the present invention also relates to a method of treating B-cell lymphoma, colorectal cancer, melanoma, head and neck squamous carcinoma, or breast cancer (e.g., triple negative breast carcinoma), by administering: (i) a therapeutically effective amount of an oncolytic virus (e.g., talimogene laherparepvec); and (ii) a therapeutically effective amount of a CTLA-4 blocker (e.g., an anti-CTLA-4 antibody such as, e.g., ipilimumab). In some embodiments, the cancer is a metastatic cancer. In another embodiment, the present invention relates to a method of treating B-cell lymphoma, colorectal cancer, melanoma, head and neck squamous carcinoma, or breast cancer (e.g., triple negative breast carcinoma), by administering: (i) a therapeutically effective amount of an oncolytic virus (e.g., talimogene laherparepvec); and (ii) a therapeutically effective amount of a PD-L1 blocker (e.g., an anti-PD-L1 antibody such as, e.g., atezolizumab). In other embodiments, the present invention relates to a method of treating B-cell lymphoma, colorectal cancer, melanoma, head and neck squamous carcinoma, or breast cancer (e.g., triple negative breast carcinoma), by administering: (i) a therapeutically effective amount of an oncolytic virus (e.g., talimogene laherparepvec); and (ii) a therapeutically effective amount of a PD-1 blocker (e.g., an anti-PD-1 antibody such as, e.g., nivolumab or pembrolizumab).

The present invention also relates to a method of treating B-cell lymphoma by administering: (i) a therapeutically effective amount of an oncolytic virus; and (ii) a therapeutically effective amount of a GITR agonist. In some embodiments, the cancer is metastatic B-cell lymphoma. In particular embodiments, the GITR agonist is: AMG 228 (also referred to as 9H6v3), TRX518, MEDI1873, or MK-4166. See, PCT publication no. WO2015031667 and U.S. Pat. No. 9,464,139, both of which are hereby incorporated by reference in their entirety. In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. The herpes simplex virus 1 may also be modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

The present invention further relates to a therapeutically effective amount of an oncolytic virus for use in treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma. In some embodiments, the cancer is a metastatic cancer. In yet another aspect, the present invention relates to a pharmaceutical composition for use in a method of treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma, wherein the pharmaceutical composition comprises an oncolytic virus. In such embodiments, the oncolytic virus may be a herpes simplex virus. The herpes simplex virus may be a herpes simplex virus 1. In some embodiments, the herpes simplex virus 1 is modified such that: (i) it does not contain an intact ICP34.5 gene. In other embodiments, the herpes simplex virus 1 is modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

In other aspects, the present invention relates to a therapeutically effective amount of an oncolytic virus and a checkpoint inhibitor for use in treating B-cell lymphoma (e.g., diffuse large B-cell lymphoma), colorectal cancer, melanoma, head and neck squamous carcinoma, or breast cancer (e.g., triple negative breast carcinoma). In some embodiments, the cancer is a metastatic cancer. In another embodiment, the present invention relates to a pharmaceutical composition for use in a method of treating B-cell lymphoma (e.g., diffuse large B-cell lymphoma), colorectal cancer, melanoma, head and neck squamous carcinoma, or breast cancer (e.g., triple negative breast carcinoma), wherein the pharmaceutical composition comprises a therapeutically effective amount of an oncolytic virus and a checkpoint inhibitor. In such embodiments, the checkpoint inhibitor is a CTLA-4 blocker (e.g., an anti-CTLA-4 antibody). In particular embodiments, the anti-CTLA-4 antibody is ipilimumab. In some embodiments, the checkpoint inhibitor is a PD-L1 blocker (e.g., an anti-PD-L1 antibody). In particular embodiments, the anti-PD-L1 antibody is atezolizumab. In some embodiments, the checkpoint inhibitor is a PD-1 blocker (e.g., an anti-PD-1 antibody). In particular embodiments, the anti-PD-1 antibody is nivolumab or pembrolizumab. In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. In other embodiments, the herpes simplex virus 1 is modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

In addition, the present invention relates to a therapeutically effective amount of an oncolytic virus (e.g., talimogene laherparepvec) and a CTLA-4 blocker (e.g., an anti-CTLA-4 antibody such as, e.g., ipilimumab) for use in treating B-cell lymphoma (e.g., diffuse large B-cell lymphoma), colorectal cancer, melanoma, head and neck squamous carcinoma, or breast cancer (e.g., triple negative breast carcinoma). In some embodiments, the cancer is a metastatic cancer. In another embodiment, the present invention relates to a therapeutically effective amount of an oncolytic virus (e.g., talimogene laherparepvec) and a PD-L1 blocker (e.g., an anti-PD-L1 antibody such as, e.g., atezolizumab) for use in treating B-cell lymphoma (e.g., diffuse large B-cell lymphoma), colorectal cancer, melanoma, head and neck squamous carcinoma, or breast cancer (e.g., triple negative breast carcinoma). In another embodiment, the present invention relates to a therapeutically effective amount of an oncolytic virus (e.g., talimogene laherparepvec) and a PD-1 blocker (e.g., an anti-PD-1 antibody such as, e.g., nivolumab, pembrolizumab). In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. In other embodiments, the herpes simplex virus 1 is modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

In another embodiment, the present invention relates to a pharmaceutical composition for use in a method of treating B-cell lymphoma (e.g., diffuse large B-cell lymphoma), colorectal cancer, melanoma, head and neck squamous carcinoma, or breast cancer (e.g., triple negative breast carcinoma), wherein the pharmaceutical composition comprises a therapeutically effective amount of an oncolytic virus (e.g., talimogene laherparepvec) and a CTLA-4 blocker (e.g., an anti-CTLA-4 antibody such as, e.g., ipilimumab). In some embodiments, the cancer is a metastatic cancer. In another embodiment, the present invention relates to a pharmaceutical composition for use in a method of treating B-cell lymphoma (e.g., diffuse large B-cell lymphoma), colorectal cancer, melanoma, head and neck squamous carcinoma, or breast cancer (e.g., triple negative breast carcinoma), wherein the pharmaceutical composition comprises a therapeutically effective amount of an oncolytic virus (e.g., talimogene laherparepvec) and a PD-L1 blocker (e.g., an anti-PD-L1 antibody such as, e.g., atezolizumab). In another embodiment, the present invention relates to a pharmaceutical composition for use in a method of treating B-cell lymphoma (e.g., diffuse large B-cell lymphoma), colorectal cancer, melanoma, head and neck squamous carcinoma, or breast cancer (e.g., triple negative breast carcinoma), wherein the pharmaceutical composition comprises a therapeutically effective amount of an oncolytic virus (e.g., talimogene laherparepvec) and a PD-1 blocker (e.g., an anti-PD-1 antibody such as, e.g., nivolumab or pembrolizumab). In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. In other embodiments, the herpes simplex virus 1 is modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

### BRIEF DESCRIPTION OF THE DRAWINGS

FIG. 1 shows the effect of intratumoral administration of talimogene laherparepvec on A-673 Ewing sarcoma tumor volume in Balb/c nude mice.

FIG. 2 shows the effect of intratumoral administration of talimogene laherparepvec on SK-N-AS neuroblastoma tumor volume in Balb/c nude mice.

FIG. 3 shows the effect of intratumoral administration of talimogene laherparepvec on G-401 rhabdoid tumor volume in Balb/c nude mice.

FIG. 4 shows the effect of intratumoral administration of talimogene laherparepvec on SJSA-1 osteosarcoma tumor volume in Balb/c nude mice.

FIG. 5 shows the effect of intratumoral administration of talimogene laherparepvec on SJCRH30 rhabdomyosarcoma tumor volume in Balb/c nude mice.

FIG. 6 shows the degree of cell growth inhibition achieved by increasing concentrations of talimogene laherparepvec in the WSU-NHL (GCB subtype) and TMD8 (ABC subtype) DLBCL cell lines.

FIG. 7 shows the degree of cell growth inhibition achieved by increasing concentrations of talimogene laherparepvec in the HCT-116 (colorectal) and SK-MEL-5 (melanoma) cell lines.

FIG. 8 shows the degree of cell growth inhibition achieved by increasing concentrations of talimogene laherparepvec in the HUT-78 (CTCL) and RPMI 8226 (multiple myeloma) cell lines.

FIG. 9 shows the degree of cell growth inhibition achieved by increasing concentrations of talimogene laherparepvec in the CT-26 and MC-38 (colorectal) cell lines.

FIG. 10a shows the effect on volume of injected tumors in A20 tumor bearing animals with OncoVex^{mGM-CSF} at three doses: 3×10⁴ PFU, 3×10⁵ PFU, and 3×10⁶ PFU. FIG. 10b shows the effect on volume of uninjected (contralateral) tumors in A20 tumor bearing animals with OncoVex^{mGM-CSF} at three doses: 3×10⁴ PFU, 3×10⁵ PFU, and 3×10⁶ PFU. FIG. 10c shows the effect on median survival of A20 tumor bearing animals treated with OncoVex^{mGM-CSF} at three doses: 3×10⁴ PFU, 3×10⁵ PFU, and 3×10⁶ PFU. FIGS. 10d and 10e show the effect of administration of OncoVex^{mGM-CSF}, anti-CTLA-4 mAb, and a combination of OncoVex^{mGM-CSF} and anti-CTLA-4 mAb on mouse bodyweight. FIG. 10f shows the effect on volume of injected tumors in neuro2a neuroblastoma tumor-bearing mice with OncoVex^{mGM-CSF} at three doses: 5×10⁴ PFU, 5×10⁵ PFU, and 5×10⁶ PFU. FIG. 10g shows the effect on median survival of neuro2a neuroblastoma tumor-bearing mice treated with OncoVex^{mGM-CSF} at three doses: 5×10⁴ PFU, 5×10⁵ PFU, and 5×10⁶ PFU. FIG. 10h shows the effect on volume of injected (treated) and uninjected (contralateral/untreated) tumors in neuro2a neuroblastoma tumor-bearing mice with OncoVex^{mGM-CSF} at 5×10⁶ PFU. FIG. 10i shows the effect on median survival of neuro2a neuroblastoma tumor-bearing mice treated with OncoVex^{mGM-CSF} at 5×10⁶ PFU.

FIG. 11a shows the effect of treatment of A20 tumor bearing animals with OncoVex^{mGM-CSF}, an anti-CTLA-4 mAb, or the combination of OncoVex^{mGM-CSF} with an anti-CTLA-4 mAb on the volume of directly injected tumors and uninjected (contralateral) tumors. FIG. 11b shows the effect of treatment of A20 tumor bearing animals with OncoVex^{mGM-CSF}, an anti-CTLA-4 mAb, or the combination of OncoVex^{mGM-CSF} with an anti-CTLA-4 mAb on median survival of the mice. FIG. 11c shows the effect of treatment of A20 tumor bearing animals with OncoVex^{mGM-CSF}, an anti-PD-L1 mAb, or the combination of OncoVex^{mGM-CSF} with an anti-PD-L1 mAb on the volume of directly injected tumors and uninjected (contralateral) tumors. FIG. 11d shows the effect of treatment of A20 tumor bearing animals with OncoVex^{mGM-CSF}, an anti-PD-L1 mAb, or the combination of OncoVex^{mGM-CSF} with an anti-PD-L1 mAb on median survival of the mice.

FIG. 12a shows the effect of treatment of CT-26 tumor bearing animals with OncoVex^{mGM-CSF}, anti CTLA-4 mAb, or the combination of OncoVex^{mGM-CSF} and an anti CTLA-4 mAb on the volume of directly injected tumors and uninjected (contralateral) tumors. FIG. 12b shows the effect of treatment of CT-26 tumor bearing animals with OncoVex^{mGM-CSF}, anti CTLA-4 mAb, or the combination of OncoVex^{mGM-CSF} and an anti CTLA-4 mAb on median survival of the mice. FIG. 12c shows the effect of treatment of CT-26 tumor bearing animals with OncoVex^{mGM-CSF}, anti-PD-L1 mAb, or the combination of OncoVex^{mGM-CSF} and an anti-PD-L1 mAb on the volume of directly injected tumors and uninjected (contralateral) tumors. FIG. 12d shows the effect of treatment of CT-26 tumor bearing animals with OncoVex^{mGM-CSF}, anti-PD-L1 mAb, or the combination of OncoVex^{mGM-CSF} and an anti-PD-L1 mAb on median survival of the mice. FIGS. 12e and 12f show the quantification of systemic (splenic) anti-AH1 CD8⁺ T-cells by ELISpot or by dextramer staining using FACS of CT-26 tumor bearing mice treated with OncoVex^{mGM-CSF}, CTLA-4 blockade, or the combination of OncoVex^{mGM-CSF} and CTLA-4 blockade. FIG. 12g shows the quantification of local (tumor) anti-AH1 CD8⁺ T-cells of CT-26 tumor bearing mice treated with OncoVex^{mGM-CSF}, CTLA-4 blockade, or the combination of OncoVex^{mGM-CSF} and CTLA-4 blockade.

FIG. 13a shows the effect of treatment of B16F10 Nectin 1 tumor bearing mice with a control, OncoVex^{mGM-CSF}, CTLA-4 blockade, or the combination of OncoVex^{mGM-CSF} and CTLA-4 blockade on the volume of injected tumors. FIG. 13b shows the assessment of lung metastasis burden on tumor bearing mice (demonstrated by the number of lung metastases) after treatment with a control, OncoVex^{mGM-CSF}, CTLA-4 blockade, or the combination of OncoVex^{mGM-CSF} and CTLA-4 blockade. FIG. 13c shows the effect of treatment with control or the combination of OncoVex^{mGM-CSF} and CTLA-4 blockade on median survival of tumor bearing mice. FIG. 13d shows that macrophages were prominent both in the tumor and in dense cellular infiltrates at the tumor periphery while B cells remained exclusively at the tumor periphery after treatment with control, OncoVex^{mGM-CSF}, CTLA-4 blockade, or the combination of OncoVex^{mGM-CSF} and CTLA-4 blockade.

FIG. 14 shows the effect of treatment of 4T1 tumor bearing mice with control or OncoVex^{mGM-CSF} on the volume of injected tumors.

FIG. 15a shows the effect of treatment of A20 tumor bearing animals with OncoVex^{mGM-CSF}, anti GITR mAb, or the combination of OncoVex^{mGM-CSF} and an anti GITR mAb on the volume of directly injected tumors and uninjected (contralateral) tumors. FIG. 15b shows the effect of treatment of A20 tumor bearing animals with OncoVex^{mGM-CSF}, anti GITR mAb, or the combination of OncoVex^{mGM-CSF} and an anti GITR mAb on median survival of the mice.

FIG. 16a shows the effect of administration of OncoVex^{mGM-CSF}, anti-PD-1 mAb, and combinations of OncoVex^{mGM-CSF} and anti-PD-1 mAb on mouse body weight. FIG. 16b shows the effect of treatment of MC-38 tumor bearing animals with OncoVex^{mGM-CSF}, anti-PD-1 mAb, or the combination of OncoVex^{mGM-CSF} and an anti-PD-1 mAb on the volume of directly injected tumors and uninjected (contralateral) tumors.

FIG. 17 shows the effect of treatment of MC-38 tumor bearing animals with OncoVex^{mGM-CSF}, anti-PD-L1 mAb, or the combination of OncoVex^{mGM-CSF} and an anti-PD-L1 mAb on the volume of directly injected tumors and uninjected (contralateral) tumors.

FIG. 18 shows the effect of treatment of B16F10 tumor bearing animals with OncoVex^{mGM-CSF}, anti-PD-1 mAb, or the combination of OncoVex^{mGM-CSF} and an anti-PD-1 mAb on the volume of directly injected tumors.

### DETAILED DESCRIPTION

The section headings used herein are for organizational purposes only and are not to be construed as limiting the subject matter described. All references cited within the body of this specification are expressly incorporated by reference in their entirety.

Standard techniques may be used for recombinant DNA, oligonucleotide synthesis, tissue culture and transformation, protein purification, etc. Enzymatic reactions and purification techniques may be performed according to the manufacturer's specifications or as commonly accomplished in the art or as described herein. The following procedures and techniques may be generally performed according to conventional methods well known in the art and as described in various general and more specific references that are cited and discussed throughout the specification. See, e.g., Sambrook et al., 2001, Molecular Cloning: A Laboratory Manuel, 3^{rd} ed., Cold Spring Harbor Laboratory Press, cold Spring Harbor, N.Y., which is incorporated herein by reference for any purpose. Unless specific definitions are provided, the nomenclature used in connection with, and the laboratory procedures and techniques of, analytic chemistry, organic chemistry, and medicinal and pharmaceutical chemistry described herein are those well-known and commonly used in the art. Standard techniques may be used for chemical synthesis, chemical analyses, pharmaceutical preparation, formulation, and delivery and treatment of patients.

### Oncolytic Viruses

As discussed herein, the present invention demonstrates that oncolytic viruses are capable of generating anti-tumor effects in a variety of tumor types either alone, or in combination with checkpoint inhibitors. A striking benefit of the oncolytic viruses of the present invention is that, compared to, e.g., chemotherapy, the anti-tumor effects are accompanied by less severe/negative side effects. For example, in one embodiment, the present invention relates to the use of oncolytic viruses in the treatment of cancer. In another embodiment, the present invention relates to the use of oncolytic viruses to treat Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma. In another embodiment, the present invention relates to the use of a combination of an oncolytic virus and a checkpoint inhibitor to treat B-cell lymphoma, colorectal cancer, melanoma, head and neck squamous carcinoma, or breast cancer (e.g., triple negative breast carcinoma.

In some embodiments, the oncolytic virus is a herpes simplex virus. The herpes simplex virus may be a herpes simplex virus 1. In some embodiments, the herpes simplex virus 1 is modified such that: (i) it does not contain an intact ICP34.5 gene. In other embodiments, the herpes simplex virus 1 is modified such that: (i) it does not contain an intact ICP34.5 gene; and (ii) it does not contain an intact ICP47 gene. In yet other embodiments, the herpes simplex virus 1 is modified such that: (i) it does not contain an intact ICP34.5 gene; (ii) it does not contain an intact ICP47 gene; and (iii) it contains a gene encoding GM-CSF (e.g., human GM-CSF). In a particular embodiment, the oncolytic virus is talimogene laherparepvec.

Talimogene laherparepvec, HSV-1 [strain JS1] ICP34.5-/ICP47-/hGM-CSF, (previously known as OncoVex^{GM-CSF}), is an intratumorally delivered oncolytic immunotherapy comprising an immune-enhanced HSV-1 that selectively replicates in solid tumors. (Lui et al., Gene Therapy, 10:292-303, 2003; U.S. Pat. No. 7,223,593 and U.S. Pat. No. 7,537,924). The HSV-1 was derived from strain JS1 as deposited at the European collection of cell cultures (ECAAC) under accession number 01010209. In talimogene laherparepvec, the HSV-1 viral genes encoding ICP34.5 have been functionally deleted. Functional deletion of ICP34.5, which acts as a virulence factor during HSV infection, limits replication in non-dividing cells and renders the virus non-pathogenic. The safety of ICP34.5-functionally deleted HSV has been shown in multiple clinical studies (MacKie et al, Lancet 357: 525-526, 2001; Markert et al, Gene Ther 7: 867-874, 2000; Rampling et al, Gene Ther 7:859-866, 2000; Sundaresan et al, J. Virol 74: 3822-3841, 2000; Hunter et al, J Virol Aug; 73(8): 6319-6326, 1999). In addition, ICP47 (which blocks viral antigen presentation to major histocompatibility complex class I and II molecules) has been functionally deleted from talimogene laherparepvec. Functional deletion of ICP47 also leads to earlier expression of US11, a gene that promotes virus growth in tumor cells without decreasing tumor selectivity. As used herein, the “lacking a functional” viral gene means that the gene(s) is partially or completely deleted, replaced, rearranged, or otherwise altered in the herpes simplex genome such that a functional viral protein can no longer be expressed from that gene by the herpes simplex virus. The coding sequence for human GM-CSF, a cytokine involved in the stimulation of immune responses, has been inserted into the viral genome (at the two former sites of the ICP34.5 genes) of talimogene laherparepvec. The insertion of the gene encoding human GM-CSF is such that it replaces nearly all of the ICP34.5 gene, ensuring that any potential recombination event between talimogene laherparepvec and wild-type virus could only result in a disabled, non-pathogenic virus and could not result in the generation of wild-type virus carrying the gene for human GM-CSF. The HSV thymidine kinase (TK) gene remains intact in talimogene laherparepvec, which renders the virus sensitive to anti-viral agents such as acyclovir. Therefore, acyclovir can be used to block talimogene laherparepvec replication, if necessary.

Examples of additional HSV genes that can be modified include ICP6, the large subunit of ribonucleotide reductase, involved in nucleotide metabolism and viral DNA synthesis in non-dividing cells but not in dividing cells. Thymidine kinase, responsible for phosphorylating acyclovir to acyclovir-monophosphate, virion trans-activator protein vmw65, glycoprotein H, vhs, ICP43, and immediate early genes encoding ICP4, ICP27, ICP22 and/or ICP0, may also modified.

Modifications may also be made to alter the timing of expression of herpes simplex virus genes. For example, US11 can be expressed as an early gene by placing the US11 gene under the Us12 promoter, Mulvey et al. (1999) J Virology, 73:4, 3375-3385, U.S. Pat. No. 5,824,318, Mohr & Gluzman (1996) EMBO 15: 4759-4766.

As would be appreciated by those of skill in the art, heterologous genes, such as those encoding human GM-CSF, can be inserted into the HSV viral genome, and viral genes, such as ICP34.5 and ICP47, can be functionally deleted using homologous recombination with plasmid DNA.

Talimogene laherparepvec produces a direct oncolytic effect by replication of the virus in the tumor, and induction of an anti-tumor immune response enhanced by the local expression of GM-CSF and the release of tumor-derived antigens via lysis. Since many cancers are present as primary and secondary (i.e., metastasized) tumors in patients, this dual activity is beneficial as a therapeutic treatment. The intended clinical effects include the destruction of injected tumors, the destruction of local, locoregional, and distant uninjected tumors, a reduction in the development of new metastases, a reduction in the rate of overall progression and of the relapse rate following the treatment of initially present disease, and prolonged overall survival.

As used herein, the terms “patient” or “subject” are used interchangeably and mean a mammal, including, but not limited to, a human or non-human mammal, such as a bovine, equine, canine, ovine, or feline. Preferably, the patient is a human.

Talimogene laherparepvec and OncoVex^{mGM-CSF} (an HSV-1 virus with the same genetic modifications as talimogene laherparepvec, except that human GM-CSF is replaced with murine GM-CSF) have been tested for efficacy in a variety of in vitro (cell line) and in vivo murine tumor models and has been shown to eradicate tumors or substantially inhibit their growth at doses comparable to those used in clinical studies. Nonclinical evaluation has also confirmed that GM-CSF enhances the immune response generated, enhancing both injected and uninjected tumor responses, and that increased surface levels of MHC class I molecules result from the deletion of ICP47. Talimogene laherparepvec has been injected into normal and tumor-bearing mice to assess its safety. In general, the virus has been well tolerated, and doses up to 1×10⁸ PFU/dose have given no indication of any safety concerns. (See, for example, Liu et al., Gene Ther 10: 292-303, 2003)

Clinical studies have been or are being conducted in several advanced tumor types with over 400 subjects treated with talimogene laherparepvec (see, for example, Hu et al., Clin Can Res 12: 6737-6747, 2006; Harrington et al., J Clin Oncol. 27(15a):abstract 6018, 2009; Kaufman et al., Ann Surgic Oncol. 17: 718-730, 2010; Kaufman and Bines, Future Oncol. 6(6): 941-949, 2010). Clinical data indicate that talimogene laherparepvec has the potential to provide overall clinical benefit to patients with advanced melanoma. In particular, a high rate of complete response was achieved in stage IIIc to IV melanoma (Scenzer et al., J. Clin. Oncol. 271(12):907-913, 2009). In addition, responses were observed in both injected and uninjected sites, including visceral sites.

The viruses of the invention may also be derived from a herpes simplex 2 (HSV-2) strain, or from a derivative thereof. Derivatives include inter-type recombinants containing DNA from HSV-1 and HSV-2 strains. Such inter-type recombinants are described in the art, for example in Thompson et al., (1998) Virus Genes 1(3); 275 286, and Meignier et al., (1998) J. Infect. Dis. 159; 602 614.

Herpes simplex virus strains may be derived from clinical isolates. Such strains are isolated from infected individuals, such as those with recurrent cold sores. Clinical isolates may be screened for a desired ability or characteristic, such as enhanced replication in tumor and/or other cells in vitro and/or in vivo in comparison to standard laboratory strains, as described in U.S. Pat. Nos. 7,063,835 and 7,223,593, each of which are incorporated by reference in their entirety. In one embodiment the herpes simplex virus is a clinical isolate from a recurrent cold sore.

Herpes simplex virus 1 virus strains include, but are not limited to, strain JS1, strain 17+, strain F, and strain KOS, strain Patton.

Further examples of modified herpes simplex viruses include, but are not limited to, Seprehvir™ (HSV1716) strain 17+ of herpes simplex virus type 1 having a deletion of 759 bp located within each copy of the BamHI s fragment (0 to 0-02 and 0-81 to 0.83 map units) of the long repeat region of the HSV genome, removing one complete copy of the 18 bp DR˜ element of the ‘a’ sequence and terminates 1105 bp upstream of the 5′ end of immediate early (1E) gene 1, see MacLean et al., (1991) Journal of General Virology 79:631-639).

G207, an oncolytic HSV-1 derived from wild-type HSV-1 strain F having deletions in both copies of the major determinant of HSV neurovirulence, the ICP 34.5 gene, and an inactivating insertion of the E. coli lacZ gene in UL39, which encodes the infected-cell protein 6 (ICP6), see Mineta et al. (1995) Nat Med. 1:938-943.

OrienX010, a herpes simplex virus with deletion of both copies of γ34.5 and the ICP47 genes as well as an interruption of the ICP6 gene and insertion of the human GM-CSF gene, see Liu et al., (2013) World Journal of Gastroenterology 19(31):5138-5143.

NV1020, a herpes simples virus with the joint region of the long (L) and short (S) regions is deleted, including one copy of ICP34.5, UL24, and UL56.34,35. The deleted region was replaced with a fragment of HSV-2 US DNA (US2, US3 (PK), gJ, and gG), see Todo, et al. (2001) Proc Natl Acad Sci USA. 98:6396-6401.

M032, a herpes simplex virus with deletion of both copies of the ICP34.5 genes and insertion of interleukin 12, see Cassady and Ness Parker, (2010) The Open Virology Journal 4:103-108.

ImmunoVEX HSV-2, is a herpes simplex virus (HSV-2) having functional deletions of the genes encoding vhs, ICP47, ICP34.5, UL43 and USS.

OncoVex^{GALV/CD}, is also derived from HSV-1 strain JS1 with the genes encoding ICP34.5 and ICP47 having been functionally deleted and the gene encoding cytosine deaminase and gibbon ape leukemia fusogenic glycoprotein inserted into the viral genome in place of the ICP34.5 genes.

Additional examples of modified herpes simplex viruses include G47delta, G47delta IL-12, ONCR-001, OrienX-010, NSC 733972, HF-10, BV-2711, JX-594, Myb34.5, AE-618, Brainwel™, and Heapwel™.

Herpes virus strains and how to make such strains are also described in U.S. Pat. Nos. 5,824,318; 6,764,675; 6,770,274; 7,063,835; 7,223,593; 7,749,745; 7,744,899; 8,273,568; 8,420,071; 8,470,577; WIPO Publication Numbers: WO199600007; WO199639841; WO199907394; WO200054795; WO2006002394; WO201306795; Chinese Patent Numbers: CN128303, CN10230334 and CN 10230335; Varghese and Rabkin, (2002) Cancer Gene Therapy 9:967-97 and Cassady and Ness Parker, (2010) The Open Virology Journal 4:103-108, each of which is incorporated herein by reference.

### Checkpoint Inhibitors

Immune checkpoints are proteins which regulate some types of immune system cells, such as T cells (which play a central role in cell-mediated immunity). Although immune checkpoints aid in keeping immune responses in check, they can also keep T cells from killing cancer cells. Immune checkpoint inhibitors (or simply “checkpoint inhibitors”) can block immune checkpoint protein activity, releasing the “brakes” on the immune system, and allowing T cells to better kill cancer cells.

As used herein, the term “immune checkpoint inhibitor” or “checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2 (Pardoll, Nature Reviews Cancer 12: 252-264, 2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or can be derived from antibodies.

Checkpoint inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PD-L1, PD-L2, PD-1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics or small molecules, that bind to and block or inhibit the activity of one or more of CTLA-4, PD-L1, PD-L2, PD-1, BTLA, HVEM, TIM3, GAL9, LAG3, VISTA, KIR, 2B4, CD 160 and CGEN-15049.

Cytotoxic T-lymphocyte-associated protein 4 (CTLA-4) is an immune checkpoint molecule that down-regulates pathways of T-cell activation. CTLA-4 is a negative regulator of T-cell activation. Blockade of CTLA-4 has been shown to augment T-cell activation and proliferation. The combination of the herpes simplex virus and the anti-CTLA-4 antibody is intended to enhance T-cell activation through two different mechanisms in order to augment the anti-tumor immune response to tumor antigen released following the lytic replication of the virus in the tumor. Therefore, the combination of the herpes simplex virus and the anti-CTLA-4 antibody may enhance the destruction of the injected and un-injected/distal tumors, improve overall tumor response, and extend overall survival, in particular where the extension of overall survival is compared to that obtained using an anti-CTLA-4 antibody alone.

Programmed cell death protein 1 (PD-1) is a 288 amino acid cell surface protein molecule expressed on T cells and pro-B cells and plays a role in their fate/differentiation. PD-1's two ligands, PD-L1 and PD-L2, are members of the B7 family. PD-1 limits the activity of T cells in peripheral tissues at the time of an inflammatory response to infection and to limit autoimmunity PD-1 blockade in vitro enhances T-cell proliferation and cytokine production in response to a challenge by specific antigen targets or by allogeneic cells in mixed lymphocyte reactions. A strong correlation between PD-1 expression and response was shown with blockade of PD-1 (Pardoll, Nature Reviews Cancer, 12: 252-264, 2012). PD-1 blockade can be accomplished by a variety of mechanisms including antibodies that bind PD-1 or PD-L1.

Programmed death-ligand 1 (PD-L1) also referred to as cluster of differentiation 274 (CD274) or B7 homolog 1 (B7-H1) is a protein encoded by the CD274 gene. See, Entrez Gene: CD274 CD274 molecule. PD-L1, a 40 kDa type 1 transmembrane protein that plays a role in suppressing the immune system, binds to its receptor (PD-1) found on activated T cells, B cells, and myeloid cells, to modulate cell activation or inhibition. See, Chemnitz et al., Journal of Immunology, 173 (2):945-54 (2004).

Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Also included are B7 inhibitors, such as B7-H3 and B7-H4 inhibitors (e.g., the anti-B7-H3 antibody MGA271 (Loo et al., 2012, Clin. Cancer Res. July 15 (18) 3834). Another checkpoint inhibitor is TIM3 (T-cell immunoglobulin domain and mucin domain 3) (Fourcade et al., 2010, J. Exp. Med. 207:2175-86 and Sakuishi et al., 2010, J. Exp. Med. 207:2187-94).

As described further herein, in one aspect, the present invention relates to the use of combinations of oncolytic viruses and checkpoint inhibitors for the treatment of cancers. In another aspect, the present invention relates to pharmaceutical compositions comprising the combination of the oncolytic viruses and checkpoint inhibitors.

Thus, in one aspect of the present invention, the checkpoint inhibitor is a blocker or inhibitor of CTLA-4, PD-1, PD-L1, or PD-L2. In some embodiments, the checkpoint inhibitor is a blocker or inhibitor of CTLA-4 such as tremelimumab, ipilimumab (also known as 10D1, MDX-D010), BMS-986249, AGEN-1884, and anti-CTLA-4 antibodies described in U.S. Pat. Nos. 5,811,097; 5,811,097; 5,855,887; 6,051,227; 6,207,157; 6,682,736; 6,984,720; and 7,605,238, each of which is incorporated herein by reference. In some embodiments, the checkpoint inhibitor is a blocker or inhibitor of PD-L1 or PD-1 (e.g., a molecule that inhibits PD-1 interaction with PD-L1 and/or PD-L2 inhibitors) such as include pembrolizumab (anti-PD-1 antibody), nivolumab (anti-PD-1 antibody), CT-011 (anti-PD-1 antibody), CX-072 (anti-PD-L1 antibody), IO-103 (anti-PD-L1), BGB-A333 (anti-PD-L1), WBP-3155 (anti-PD-L1), MDX-1105 (anti-PD-L1), LY-3300054 (anti-PD-L1), KN-035 (anti-PD-L1), FAZ-053 (anti-PD-L1), CK-301 (anti-PD-L1), AK-106 (anti-PD-L1), M-7824 (anti-PD-L1), CA-170 (anti-PD-L1), CS-1001 (anti-PD-L1 antibody); SHR-1316 (anti-PD-L1 antibody); BMS 936558 (anti-PD-1 antibody), BMS-936559 (anti-PD-1 antibody), atezolizumab (anti-PD-L1 antibody), AMP 224 (a fusion protein of the extracellular domain of PD-L2 and an IgG1 antibody designed to block PD-L2/PD-1 interaction), MEDI4736 (durvalumab; anti PD-L1 antibody), MSB0010718C (anti-PD-L1 antibody), and those described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699, each of which is incorporated herein by reference. Additional anti-PD-1 antibodies include PDR-001; SHR-1210; BGB-A317; BCD-100; JNJ-63723283; PF-06801591; BI-754091; JS-001; AGEN-2034; MGD-013; LZM-009; GLS-010; MGA-012; AK-103; genolimzumab; dostarlimab; cemiplimab; IBI-308; camrelizumab; AMP-514; TSR-042; Sym-021; HX-008; and ABBV-368.

BMS 936558 is a fully human IgG4 monoclonal antibody targeting PD-1. In a phase I trial, biweekly administration of BMS-936558 in subjects with advanced, treatment-refractory malignancies showed durable partial or complete regressions. The most significant response rate was observed in subjects with melanoma (28%) and renal cell carcinoma (27%), but substantial clinical activity was also observed in subjects with non-small cell lung cancer (NSCLC), and some responses persisted for more than a year.

BMS 936559 is a fully human IgG4 monoclonal antibody that targets the PD-1 ligand PD-L1. Phase I results showed that biweekly administration of this drug led to durable responses, especially in subjects with melanoma. Objective response rates ranged from 6% to 17%) depending on the cancer type in subjects with advanced-stage NSCLC, melanoma, RCC, or ovarian cancer, with some subjects experiencing responses lasting a year or longer.

AMP 224 is a fusion protein of the extracellular domain of the second PD-1 ligand, PD-L2, and IgG1, which has the potential to block the PD-L2/PD-1 interaction. AMP-224 is currently undergoing phase I testing as monotherapy in subjects with advanced cancer.

MEDI4736 is an anti-PD-L1 antibody that has demonstrated an acceptable safety profile and durable clinical activity in this dose-escalation study. Expansion in multiple cancers and development of MEDI4736 as monotherapy and in combination is ongoing.

### GITR Agonists

Glucocorticoid-induced TNFR-related gene (GITR: TNFRSF 18), sometimes also referred to as Activation-Inducible TNFR family member (AITR), is a receptor belonging to the TNF receptor superfamily (TNFRSF). It is activated by its cognate ligand, GITR ligand (GITRL, TNFSF18). GITR is a type I transmembrane protein that contains a cysteine-rich extracellular domain, which is characteristic of TNFR family members. The cytoplasmic domain of GITR, for instance, shares close homology with certain other TNFR family members, such as 4-1BB and CD27 (Nocentini, et al., Proc. Natl. Acad. Sci., 94:6216-6221 (1997)). GITR agonist antibodies are currently being explored as a means of expanding the CD8+ T effector memory cell population while, at the same time, promoting the loss or inhibition of Tregs.

The co-stimulation of responder T cells and abrogation of the suppressor activity of regulatory T cells, means that GITR activation results in an enhanced immune response. Such activation has the potential to restore immune responses to infections and to tumors. Accordingly, molecules capable of activating GITR would be of value as immunostimulatory agents in settings in which it is desirable to trigger an enhanced immune response.

As described further herein, in one aspect, the present invention relates to the use of combinations of oncolytic viruses and GITR agonists for the treatment of cancers. In another aspect, the present invention relates to pharmaceutical compositions comprising the combination of the oncolytic viruses and GITR agonists.

In some embodiments, the GITR agonist is AMG 228 (also referred to as 9H6v3), TRX518, MEDI1873, MK-4166, BMS-986156, MK-1248, INCAGN01876, or GWN323.

TRX518 is a humanized, Fc disabled anti-GITR monoclonal antibody that blocks the interaction of GITR and has been shown to act synergistically with chemotherapeutic drugs in cancer models. TRX518 is currently being investigated in clinical trials including NCT01239134 (Stage III or IV malignant melanoma or other solid tumors), and NCT02628574 (advanced solid tumors).

MEDI1873 is a GITR agonist (a GITR ligand (GITRL) IgG1 fusion protein) with potential immunomodulating and antineoplastic activities. MEDI1873 is currently being investigated in clinical trials including NCT02583165 (advanced solid tumors).

MK-4166 is an anti GITR agonistic monoclonal antibody that has been shown to act synergistically with chemotherapeutic drugs in cancer models. MK-4166 is currently being investigated in clinical trials including NCT02132754 (in combination with pembrolizumab in advanced solid tumors).

BMS-986156 is an anti GITR agonistic monoclonal antibody. BMS-986156 is currently being investigated in clinical trials including NCT02598960 (as a monotherapy and in combination with nivolumab in subjects with advanced solid tumors).

MK-1248 is an anti GITR agonistic monoclonal antibody. MK-1248 is currently being investigated in clinical trials including NCT02553499 (as a monotherapy and in combination with pembrolizumab in subjects with advanced solid tumors).

INCAGN01876 is an anti GITR agonistic monoclonal antibody. INCAGN01876 is currently being investigated in clinical trials including NCT02697591 (in subjects with advanced or metastatic solid tumors).

GWN323 is an anti GITR agonistic monoclonal antibody. GWN323 is currently being investigated in clinical trials including NCT02697591 (as a monotherapy and in combination with PDR001 in subjects with advanced cancer or lymphomas).

Methods of Treating a Disease or Disorder

The present invention also relates to methods of treating diseases or disorders, such as cancer. In some embodiments, the cancer is Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma. In other embodiments, the cancer is B-cell lymphoma (e.g., diffuse large B-cell lymphoma), colorectal cancer, melanoma, or breast cancer (e.g., triple negative breast carcinoma). In some embodiments, the cancer is a metastatic cancer.

The term “metastatic cancer” refers to a cancer that has spread from the part of the body where it started (i.e., the primary site) to other parts of the body. When cancer has spread to a new area (i.e., metastasized), it's still named after the part of the body where it started. For instance, colon cancer that has spread to the pancreas is referred to as “metastatic colon cancer to the pancreas,” as opposed to pancreatic cancer. Treatment is also based on where the cancer originated. If colon cancer spreads to the bones, it's still a colon cancer, and the relevant physician will recommend treatments that have been shown to combat metastatic colon cancer.

The present invention also relates to the use of combinations of oncolytic viruses and checkpoint inhibitors for the treatment of cancers. In some embodiments, the cancer is Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma. In other embodiments, the cancer is B-cell lymphoma (e.g., diffuse large B-cell lymphoma), colorectal cancer, melanoma, head and neck squamous carcinoma, or breast cancer (e.g., triple negative breast carcinoma). In some embodiments, the cancer is a metastatic cancer.

The present invention also relates to a method of treating diseases or disorders, such as cancer by administering: (i) a therapeutically effective amount of an oncolytic virus; and (ii) a therapeutically effective amount of a GITR agonist. In particular embodiments, the cancer is: B-cell lymphoma. In other embodiments, the GITR agonist is: AMG 228, TRX518, MEDI1873, or MK-4166.

The oncolytic virus may be any of those described herein. In some embodiments, the oncolytic virus is a herpes simplex virus (e.g., a herpes simplex virus 1). In other embodiments, the herpes simplex virus 1 is modified such that it does not contain an intact ICP34.5 gene. In other embodiments, the herpes simplex virus 1 is modified such that it does not contain an intact ICP34.5 gene, and it does not contain an intact ICP47 gene. In yet other embodiments, the herpes simplex virus 1 is modified such that it does not contain an intact ICP34.5 gene, it does not contain an intact ICP47 gene, and it contains a gene encoding GM-CSF (e.g., human GM-CSF). In a specific embodiment, the oncolytic virus is talimogene laherparepvec.

The checkpoint inhibitor can be any molecule that blocks or inhibits the inhibitory pathways of the immune system. For example, the following checkpoint molecules may be targeted for blocking or inhibition: CTLA-4, PD-L1, PD-L2, PD-1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR and various B-7 family ligands. B7 family ligands include, but are not limited to, B7-1, B7-2, B7-DC, B7-H1, B7-H2, B7-H3, B7-H4, B7-H5, B7-H6 and B7-H7. Example of checkpoint inhibitors include binding proteins (e.g., antibodies, or antigen binding fragments thereof), biologic therapeutics, or small molecules, that bind to and block or inhibit the activity of one or more of CTLA-4, PD-L1, PD-L2, PD-1, BTLA, HVEM, TIM3, GAL9, LAG3, VISTA, KIR, 2B4, CD 160 and CGEN-15049.

In some embodiments, the checkpoint inhibitor is a blocker or inhibitor of CTLA-4, PD-1, PD-L1, or PD-L2. Examples of CTLA-4 inhibitors include tremelimumab, ipilimumab (also known as 10D1, MDX-D010), BMS-986249, AGEN-1884, and anti-CTLA-4 antibodies described in U.S. Pat. Nos. 5,811,097; 5,811,097; 5,855,887; 6,051,227; 6,207,157; 6,682,736; 6,984,720; and 7,605,238, each of which is incorporated herein by reference. Examples of molecules that inhibit PD-1 interaction with PD-L1 and/or PD-L2 inhibitors include pembrolizumab (anti-PD-1 antibody), nivolumab (BMS 936558; anti-PD-1 antibody), CT-011 (anti-PD-1 antibody), BMS 936558 (anti-PD-1 antibody), BMS-936559 (anti-PD-L1 antibody), CX-072 (anti-PD-L1 antibody), IO-103 (anti-PD-L1), BGB-A333 (anti-PD-L1), WBP-3155 (anti-PD-L1), MDX-1105 (anti-PD-L1), LY-3300054 (anti-PD-L1), KN-035 (anti-PD-L1), FAZ-053 (anti-PD-L1), CK-301 (anti-PD-L1), AK-106 (anti-PD-L1), M-7824 (anti-PD-L1), CA-170 (anti-PD-L1), CS-1001 (anti-PD-L1 antibody), SHR-1316 (anti-PD-L1 antibody), atezolizumab (anti-PD-L1 antibody), AMP 224 (a fusion protein of the extracellular domain of PD-L2 and an IgG1 antibody designed to block PD-L2/PD-1 interaction), MEDI4736 (durvalumab; anti PD-L1 antibody), MSB0010718C (avelumab; anti-PD-L1 antibody), and those described in U.S. Pat. Nos. 7,488,802; 7,943,743; 8,008,449; 8,168,757; 8,217,149, and PCT Published Patent Application Nos: W003042402, WO2008156712, WO2010089411, WO2010036959, WO2011066342, WO2011159877. Additional anti-PD-1 antibodies include PDR-001; SHR-1210; BGB-A317; BCD-100; JNJ-63723283; PF-06801591; BI-754091; JS-001; AGEN-2034; MGD-013; LZM-009; GLS-010; MGA-012; AK-103; genolimzumab; dostarlimab; cemiplimab; IBI-308; camrelizumab; AMP-514; TSR-042; Sym-021; HX-008; and ABBV-368.

In particular embodiments, the present invention relates to a combination of an oncolytic virus and an anti-PD-1 antibody, an oncolytic virus and an anti-PD-L1 antibody, or an oncolytic virus and an anti-CTLA-4 antibody. In specific embodiments, the oncolytic virus is talimogene laherparepvec.

In many instances, cancer is present in patients as both a primary tumor (i.e., a tumor growing at the anatomical site where tumor progression began and proceeded to yield a cancerous mass) and as a secondary tumor or metastasis (i.e., the spread of a tumor from its primary site to other parts of the body). The oncolytic viruses of the present invention can be efficacious in treating tumors via a lytic effect and systemic immune effect. For example, in the context of talimogene laherparepvec, the virus physically lyses tumors cells causing primary tumor cell death. In addition, [1] the lysis of tumor cells releases tumor-derived antigens which are then recognized by the immune system; and [2] the production of GM-CSF aids in the induction of the anti-tumor immune response both mechanisms are thought to lead to a systemic immune response whereby the immune system can recognize and attack both the primary and secondary tumors/metastases. In embodiments where the oncolytic virus is combined with a checkpoint inhibitor, the checkpoint inhibitor is thought to further enhance the systemic immune response by enhancing priming and reducing the inhibitory effect of immune checkpoint proteins on immune system cells, such as T cells. Moreover, in embodiments where the oncolytic virus is combined with a GITR agonist, the GITR agonist is thought to further enhance the systemic immune response by expanding the CD8+ T effector memory cell population and to promote the loss or inhibition of Tregs. Accordingly, the present invention contemplates the treatment of primary tumors, metastases (i.e., secondary tumors), or both with an oncolytic virus (e.g., talimogene laherparepvec) either alone or in combination with a checkpoint inhibitor.

In some embodiments, the methods of treatment or uses described herein do not include treatment with radiation or a combination treatment with radiation. In other embodiments, the methods of treatment or uses described herein do not include treatment with chemotherapeutics (i.e., chemical agents or drugs typically small molecule compounds that are selectively destructive to malignant cells and tissues), such as cisplatin, or a combination treatment with chemotherapeutics (e.g., cisplatin). In yet other embodiments, the methods of treatment or uses described herein do not include treatment with a combination of radiation and a chemotherapeutic (e.g., cisplatin).

The methods of the present invention can be used to treat several different stages of cancer. Most staging systems include information relating to whether the cancer has spread to nearby lymph nodes, where the tumor is located in the body, the cell type (e.g., squamous cell carcinoma), whether the cancer has spread to a different part of the body, the size of the tumor, and the grade of tumor (i.e., the level of cell abnormality the likelihood of the tumor to grow and spread). For example, Stage 0 refers to the presence of abnormal cells that have not spread to nearby tissue i.e., cells that may become a cancer. Stage I, Stage II, and Stage III cancer refer to the presence of cancer. The higher the Stage, the larger the cancer tumor and the more it has spread into nearby tissues. Stage IV cancer is cancer that has spread to distant parts of the body. In some embodiments, the methods of the present invention can be used to treat metastatic cancer.

### Pharmaceutical Compositions

The present invention also relates to pharmaceutical compositions comprising oncolytic viruses, or comprising the combination of the oncolytic viruses and checkpoint inhibitors. The pharmaceutical composition may contain formulation materials for modifying, maintaining or preserving, for example, the pH, osmolarity, viscosity, clarity, color, isotonicity, odor, sterility, stability, rate of dissolution or release, adsorption, or penetration of the composition. Pharmaceutically active agents can be administered to a patient by various routes including, for example, orally or parenterally, such as intravenously, intramuscularly, subcutaneously, intraorbitally, intracapsularly, intraperitoneally, intrarectally, intracisternally, intratumorally, intravasally, intradermally or by passive or facilitated absorption through the skin using, for example, a skin patch or transdermal iontophoresis, respectively. In one embodiment, the oncolytic virus (e.g., talimogene laherparepvec) is injected into the tumor (i.e., via intratumoral injection). In another embodiment, the checkpoint inhibitor (e.g., an anti-PD-1 antibody, anti-PD-L1 antibody, or anti-CTLA-4 antibody) is administered systemically (e.g., intravenously).

One of ordinary skill in the art would be able to determine the dosage and duration of treatment according to any aspect of the present disclosure. For example, the skilled artisan may monitor patients to determine whether treatment should be started, continued, discontinued or resumed. An effective amount for a particular patient may vary depending on factors such as the condition being treated, the overall health of the patient and the method, route and dose of administration. The clinician using parameters known in the art makes determination of the appropriate dose. An effective amount of a pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding agent molecule is being used, the route of administration, and the size (body weight, body surface or organ size) and condition (the age and general health) of the patient. Accordingly, the clinician may titer the dosage and modify the route of administration to obtain the optimal therapeutic effect.

Clinical studies have demonstrated that talimogene laherparepvec can be injected directly into cutaneous, subcutaneous or nodal lesions that are visible, palpable, or can be injected with ultrasound-guidance. Thus, in one aspect, pharmaceutical compositions comprising talimogene laherparepvec are administered via intralesional injection. Talimogene laherparepvec is currently provided in 1 mL single-use vials in fixed dosing concentrations: 10⁶ pfu/mL for initial dosing and 10⁸ pfu/mL for subsequent dosing (Reske, et al. J Immunol, 2008. 180(11): p. 7525-36). The volume that is injected may vary depending on the tumor type. For example, talimogene laherparepvec is administered by intratumoral injection into injectable cutaneous, subcutaneous, and nodal tumors at a dose of up to 4.0 mL of 10⁶ plaque forming unit/mL (PFU/mL) at day 1 of week 1 followed by a dose of up to 4.0 mL of 10⁸ PFU/mL at day 1 of week 4, and every 2 weeks (±3 days) thereafter. In another embodiment, talimogene laherparepvec is administered by intratumoral injection into injectable cutaneous, subcutaneous, and nodal tumors at a dose of up to 4.0 mL of 10⁶ plaque forming unit/mL (PFU/mL) at day 1 of week 1 followed by a dose of up to 4.0 mL of 10⁷ PFU/mL at day 1 of week 4, and every 2 weeks (±3 days) thereafter. The recommended volume of talimogene laherparepvec to be injected into the tumor(s) is dependent on the size of the tumor(s) and may be determined according to the injection volume guideline in Table 1 (and as shown in patent application PCT/US2013/057542, which is incorporated herein by reference).

TABLE 1
Talimogene Laherparepvec Injection Volume
Guidelines Based on Tumor Size

| Tumor Size (longest dimension) | Maximum Injection Volume |
|---|---|
| ≥5.0 cm | 4.0 mL |
| >2.5 cm to 5.0 cm | 2.0 mL |
| >1.5 cm to 2.5 cm | 1.0 mL |
| >0.5 cm to 1.5 cm | 0.5 mL |
| ≤0.5 cm | 0.1 mL |

Generally, all reasonably injectable lesions should be injected with the maximum dosing volume available on an individual dosing occasion. On each treatment day, prioritization of injections is recommended as follows: any new injectable tumor that has appeared since the last injection; by tumor size, beginning with the largest tumor; any previously uninjectable tumor(s) that is now injectable.

Compositions of the present invention may comprise one or more additional components including a physiologically acceptable carrier, excipient or diluent. For example, the compositions may comprise one or more of a buffer, an antioxidant such as ascorbic acid, a low molecular weight polypeptide (e.g., having fewer than 10 amino acids), a protein, an amino acid, a carbohydrate such as glucose, sucrose or dextrins, a chelating agent such as EDTA, glutathione, a stabilizer, and an excipient. Acceptable diluents include, for example, neutral buffered saline or saline mixed with specific serum albumin. Preservatives such as benzyl alcohol may also be added. The composition may be formulated as a lyophilizate using appropriate excipient solutions (e.g., sucrose) as diluents.

In certain embodiments, the checkpoint inhibitor is administered in 0.01 mg/kg, 0.05 mg/kg, 0.1 mg/kg, 0.2 mg/kg, 0.3 mg/kg, 0.5 mg/kg, 0.7 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, or any combination thereof doses. In certain embodiments the checkpoint inhibitor is administered once a week, twice a week, three times a week, once every two weeks, or once every month. In certain embodiments, the checkpoint inhibitor is administered as a single dose, in two doses, in three doses, in four doses, in five doses, or in 6 or more doses.

In certain embodiments, the anti-PD-1 antibody is administered by injection (e.g., subcutaneously or intravenously) at a dose of about 1 to 30 mg/kg, e.g., about 5 to 25 mg/kg, about 10 to 20 mg/kg, about 1 to 5 mg/kg, or about 3 mg/kg. The dosing schedule can vary from e.g., once a week to once every 2, 3, or 4 weeks. In one embodiment, the anti-PD-1 antibody is administered at a dose from about 10 to 20 mg/kg every other week.

In one embodiment, the anti-PD-1 antibody molecule, e.g., nivolumab, is administered intravenously at a dose from about 1 mg/kg to 3 mg/kg, e.g., about 1 mg/kg, 2 mg/kg or 3 mg/kg, every two weeks. In one embodiment, the anti-PD-1 antibody molecule, e.g., nivolumab, is administered intravenously at a dose of about 2 mg/kg at 3-week intervals. In one embodiment, nivolumab is administered in an amount from about 1 mg/kg to 5 mg/kg, e.g., 3 mg/kg, and may be administered over a period of 60 minutes, ca. once a week to once every 2, 3 or 4 weeks.

In one embodiment, the anti-PD-1 antibody molecule, e.g., pembrolizumab, is administered intravenously at a dose from about 1 mg/kg to 3 mg/kg, e.g., about 1 mg/kg, 2 mg/kg or 3 mg/kg, every three weeks. In one embodiment, the anti-PD-1 antibody molecule, e.g., pembrolizumab, is administered intravenously at a dose of about 2 mg/kg at 3-week intervals. In another embodiment, the anti-PD-1 antibody molecule, e.g., pembrolizumab, is administered intravenously at a dose from about 100 mg/kg to 300 mg/kg, e.g., about 100 mg/kg, 200 mg/kg or 300 mg/kg, every three weeks. In one embodiment, the anti-PD-1 antibody molecule, e.g., pembrolizumab, is administered intravenously at a dose of about 200 mg/kg at 3-week intervals.

In certain embodiments, the anti-CTLA-4 antibody (e.g., ipilimumab) is administered by injection (e.g., subcutaneously or intravenously) at a dose of about 3 mg/kg IV Q3W for a maximum of 4 doses; about 3 mg/kg IV Q6W for a maximum of 4 doses; about 3 mg/kg IV Q12W for a maximum of 4 doses; about 10 mg/kg IV Q3W for a maximum of 4 doses; or about 10 mg/kg IV Q12W for a maximum of 4 doses. In certain embodiments, the anti-CTLA-4 antibody (e.g., tremelimumab) is administered by injection (e.g., subcutaneously or intravenously) at a dose of about 10 mg/kg Q4W; or about 15 mg/kg every 3 months.

In certain embodiments, the anti-PD-L1 antibody (e.g., atezolizumab) is administered by injection (e.g., subcutaneously or intravenously) at a dose of about 1200 mg IV Q3W until disease progression or unacceptable toxicity.

Thus, in one embodiment, the present invention relates to a pharmaceutical composition for use in a method of treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma, wherein the pharmaceutical composition comprises an oncolytic virus. In another embodiment, the present invention relates to a pharmaceutical composition for use in a method of treating B-cell lymphoma (e.g., diffuse large B-cell lymphoma), colorectal cancer, melanoma, head and neck squamous carcinoma, or breast cancer (e.g., triple negative breast carcinoma), wherein the pharmaceutical composition comprises a therapeutically effective amount of an oncolytic virus and a checkpoint inhibitor. In additional embodiments, the present invention relates to a pharmaceutical composition for use in a method of treating B-cell lymphoma, wherein the pharmaceutical composition comprises a therapeutically effective amount of an oncolytic virus and a GITR agonist.

In other embodiments, the present invention relates to a therapeutically effective amount of an oncolytic virus for use in treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma. In yet other embodiments, the present invention relates to a therapeutically effective amount of an oncolytic virus and a checkpoint inhibitor for use in treating B-cell lymphoma (e.g., diffuse large B-cell lymphoma), colorectal cancer, melanoma, head and neck squamous carcinoma, or breast cancer (e.g., triple negative breast carcinoma). In additional embodiments, the present invention relates to a therapeutically effective amount of an oncolytic virus and a GITR agonist for use in treating B-cell lymphoma.

In some embodiments, the oncolytic virus is a herpes simplex virus (e.g., a herpes simplex virus 1). In other embodiments, the herpes simplex virus 1 is modified such that it does not contain an intact ICP34.5 gene. In other embodiments, the herpes simplex virus 1 is modified such that it does not contain an intact ICP34.5 gene, and it does not contain an intact ICP47 gene. In yet other embodiments, the herpes simplex virus 1 is modified such that it does not contain an intact ICP34.5 gene, it does not contain an intact ICP47 gene, and it contains a gene encoding GM-CSF (e.g., human GM-CSF). In a specific embodiment, the oncolytic virus is talimogene laherparepvec.

In embodiments where the pharmaceutical composition comprises a checkpoint inhibitor, the checkpoint inhibitor may be any of those discussed herein. For example, the checkpoint inhibitor may be a CTLA-4 blocker, a PD-L1 blocker, or a PD-1 blocker. The CTLA-4 blocker may be an anti-CTLA-4 antibody such as, e.g., ipilimumab. The PD-L1 blocker may be an anti-PD-L1 antibody such as, e.g., atezolizumab. The PD-1 blocker maybe an anti-PD-1 antibody such as, e.g., nivolumab or pembrolizumab.

In some embodiments, the GITR agonist is AMG 228, TRX518, MEDI1873, or MK-4166.

In some embodiments, the pharmaceutical compositions described herein are not used in conjunction with radiation or in a combination treatment with radiation. In other embodiments, the pharmaceutical compositions described herein do not comprise chemotherapeutics (e.g., cisplatin). In yet other embodiments, the pharmaceutical compositions described herein are not used in treatment with a combination of radiation and a chemotherapeutic (e.g., cisplatin).

### Kits

In another aspect, the present invention relates to kits comprising [1] the oncolytic virus, optionally in combination with a checkpoint inhibitor; and [2] instructions for administration to patients. For example, a kit of the present invention may comprise an oncolytic virus (e.g., talimogene laherparepvec), and instructions (e.g., in a package insert or label) for treating a patient with cancer. In some embodiments, the cancer is a metastatic cancer. In another embodiment, the kit of the present invention may comprise an oncolytic virus (e.g., talimogene laherparepvec), a checkpoint inhibitor (e.g., an anti-PD-1 antibody, anti-PD-L1 antibody, or anti-CTLA-4 antibody), and instructions (e.g., in a package insert or label) for treating a patient with cancer.

In another aspect, the present invention relates to kits comprising [1] the oncolytic virus, optionally in combination with a GITR agonist; and [2] instructions for administration to patients. In other embodiments, the kit of the present invention may comprise an oncolytic virus (e.g., talimogene laherparepvec), a GITR agonist (e.g., AMG 228 (also referred to as 9H6v3), TRX518, MEDI1873, or MK-4166), and instructions (e.g., in a package insert or label) for treating a patient with cancer.

In some embodiments, the kit comprising talimogene laherparepvec comprises instructions (e.g., in a package insert or label) for administration by intratumoral injection at a dose of up to 4.0 ml of 10⁶ PFU/mL at day 1 of week 1 followed by a dose of up to 4.0 ml of 10⁸ PFU/mL at day 1 of week 4, and every 2 weeks thereafter (e.g., until complete response). In some embodiments, the kit comprising talimogene laherparepvec comprises instructions (e.g., in a package insert or label) for administration by intratumoral injection at a dose of up to 4.0 ml of 10⁶ PFU/mL at day 1 of week 1 followed by a dose of up to 4.0 ml of 10⁷ PFU/mL at day 1 of week 4, and every 2 weeks thereafter (e.g., until complete response).

In embodiments where the kit comprises an anti-PD-1 antibody, the kit comprises instructions (e.g., in a package insert or label) for intravenous administration at a doses described herein. Examples of anti-PD-1 antibodies include, pembrolizumab and nivolumab.

In embodiments where the kit comprises an anti-PD-L1 antibody, the kit comprises instructions (e.g., in a package insert or label) for intravenous administration at a doses described herein. Examples of anti-PD-L1 antibodies include, atezolizumab.

In embodiments where the kit comprises an anti-CTLA-4 antibody, the kit comprises instructions (e.g., in a package insert or label) for intravenous administration at a doses described herein. Examples of anti-CTLA-4 antibodies include, ipilimumab.

In embodiments where the kit comprises a GITR agonist, the kit comprises instructions (e.g., in a package insert or label) for intravenous administration at a doses described herein. Examples of anti-GITR antibodies include, AMG 228, TRX518, MEDI1873, or MK4166.

In another embodiment is provided a method of manufacturing the kits of the present invention.

In some embodiments, the kits described herein are not used in conjunction with radiation or in a combination treatment with radiation. In other embodiments, the kits described herein do not comprise chemotherapeutics (e.g., cisplatin). In yet other embodiments, the kits described herein are not used in treatment with a combination of radiation and a chemotherapeutic (e.g., cisplatin).

### EXAMPLES

The following examples are provided for the purpose of illustrating specific embodiments or features of the present invention and are not intended to limit its scope.

### Example 1

### Talimogene laherparepvec Exhibits Antitumor Activity Against a Range of Tumor Types in an In Vivo Mouse Model

This example demonstrates that administration of talimogene laherparepvec to tumor bearing mice leads to tumor killing.

The anti-tumor efficacy of talimogene laherparepvec was assessed in several mouse xenograft studies in Balb/c nude mice. Tumor cells (A-673 human pediatric Ewing sarcoma, SJCRH30 human pediatric rhabdomyosarcoma, G-401 human pediatric rhabdoid tumor, SK-N-AS human pediatric neuroblastoma, or SJSA-1 human pediatric osteosarcoma) were implanted into the right flank of each mouse by subcutaneous injection. In each case, 5×10⁶ to 1×10⁷ cells in 100-200 μL 50% matrigel/50% DMEM were implanted into the mice.

Tumor measurements were obtained twice weekly. Treatment with talimogene laherparepvec started when tumors reached an average of 4-6 mm in diameter. Three talimogene laherparepvec doses (5×10⁴, 5×10⁵, or 5×10⁶ PFU/dose, 50 uL dose volume) were administered three days apart by intratumoral injection. Body weights, gross clinical observations, and tumor measurements were obtained twice weekly. Animals were euthanized when the tumor weight exceeded 10% of body weight.

In these experiments, talimogene laherparepvec showed anti-tumor efficacy against all the cell lines tested, with tumor growth inhibition of 65-112% and evidence of complete regression in 3-30% of animals across the tumor types.

### Ewing Sarcoma

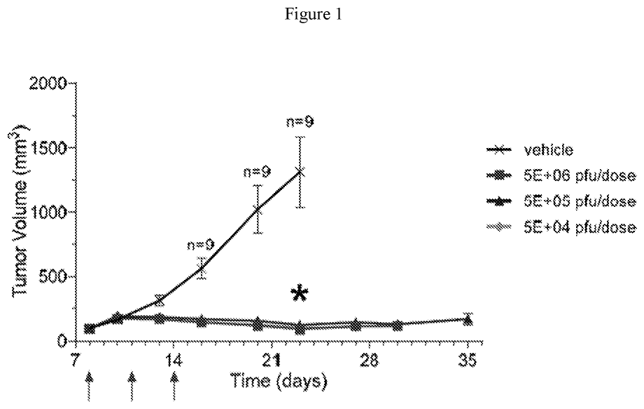

A-673 Ewing's sarcoma tumor-bearing mice were treated therapeutically with talimogene laherparepvec at 5×10⁴, 5×10⁵, or 5×10⁶ PFU/dose. Talimogene laherparepvec was administered by intratumoral injection once daily on study days 8, 11 and 14 (FIG. 1, red arrows). Tumors were measured 2-3× per week. Results are expressed as the group mean tumor volume in mm³±standard error of the mean as a function of time in days where day 0 is the day of tumor inoculation (n=10 per group except as noted for the vehicle control group on days 16, 20 and 23). The asterisk indicates p<0.0001 for all talimogene laherparepvec groups relative to vehicle control on study day 23.

Results are shown in FIG. 1.

### Neuroblastoma

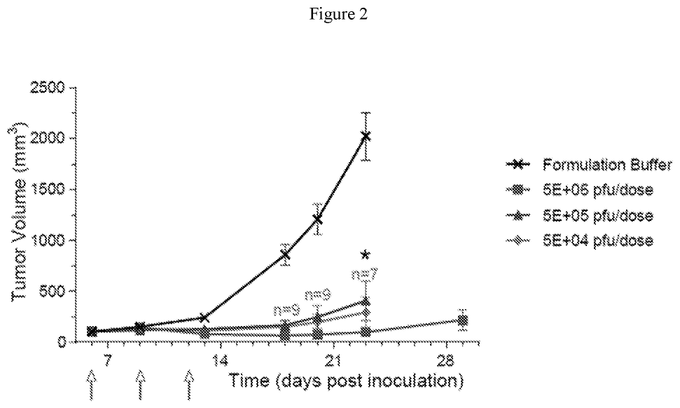

SK-N-AS neuroblastoma tumor-bearing mice were treated therapeutically with talimogene laherparepvec at 5×10⁴, 5×10⁵, or 5×10⁶ PFU/dose. Talimogene laherparepvec was administered by intratumoral injection once daily on study days 6, 9 and 12 (FIG. 2, red arrows). Tumors were measured 2-3× per week. Results are expressed as the group mean tumor volume in mm³±standard error of the mean as a function of time in days where day 0 is the day of tumor inoculation (n=10 per group except as noted for the 5×10⁴ PFU/dose group on days 18, 20 and 23). The asterisk indicates p<0.0001 for the 5×10⁶ and 5×10⁵ PFU/dose groups and p=0.0001 for the 5×10⁴ PFU/dose group relative to formulation buffer control on study day 23.

Results are shown in FIG. 2.

Rhabdoid Tumor

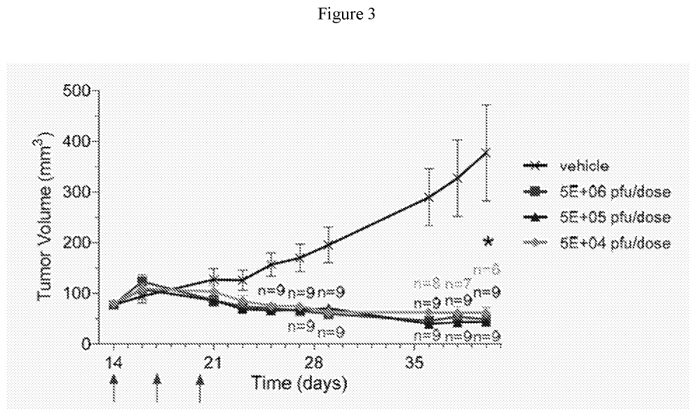

G-401 rhabdoid tumor-bearing mice were treated therapeutically with talimogene laherparepvec at 5×10⁴, 5×10⁵, or 5×10⁶ PFU/dose. Talimogene laherparepvec was administered by intratumoral injection once daily on study days 14, 17 and 20 (FIG. 3, red arrows). Tumors were measured 2-3× per week. Results are expressed as the group mean tumor volume in mm³±standard error of the mean as a function of time in days where day 0 is the day of tumor inoculation (n=10 per group except as noted). The asterisk indicates p<0.0001 for all talimogene laherparepvec dose groups relative to vehicle control on study day 40.

Results are shown in FIG. 3.

### Osteosarcoma

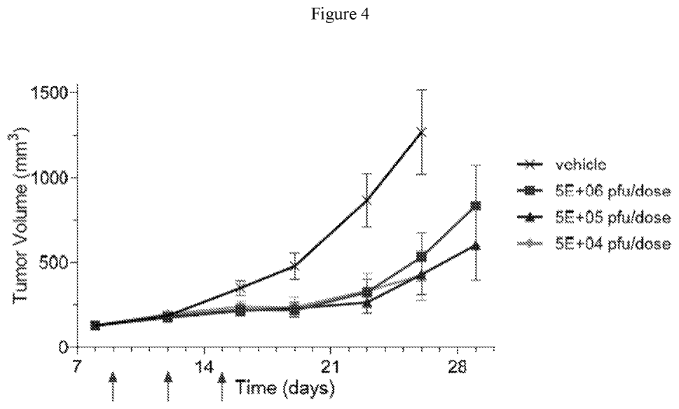

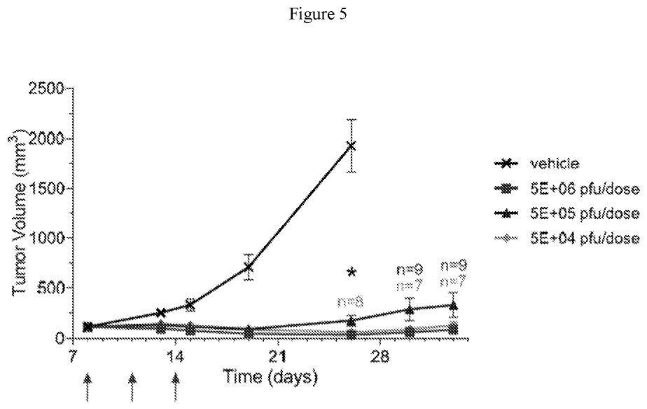

SJSA-1 osteosarcoma tumor-bearing mice were treated therapeutically with talimogene laherparepvec at 5×10⁴, 5×10⁵, or 5×10⁶ PFU/dose. Talimogene laherparepvec was administered by intratumoral injection once daily on study days 9, 12 and 15 (FIG. 4, red arrows). Tumors were measured 2-3× per week. Results are expressed as the group mean tumor volume in mm³±standard error of the mean as a function of time in days where day 0 is the day of tumor inoculation (n=10 per group).

Results are shown in FIG. 4.

### Rhabdomyosarcoma

SJCRH30 rhabdomyosarcoma tumor-bearing mice were treated therapeutically with talimogene laherparepvec at 5×10⁴, 5×10⁵, or 5×10⁶ PFU/dose. Talimogene laherparepvec was administered by intratumoral injection once daily on study days 8, 11 and 14 (FIG. 5, red arrows). Tumors were measured 2-3× per week. Results are expressed as the group mean tumor volume in mm³±standard error of the mean as a function of time in days where day 0 is the day of tumor inoculation (n=10 per group except as noted for the 5×10⁴ PFU/dose group on days 26, 30 and 33 and for the 5×10⁶ PFU/dose group on days 30 and 33). The asterisk indicates p<0.0001 for all talimogene laherparepvec dose groups relative to vehicle control on study day 26 (the last study day where all control animals were on study).

Results are shown in FIG. 5.

### Example 2

### Talimogene laherparepvec Inhibits the Growth of a Range of Human Tumor Types in Cell-Based Assays

### Diffuse Large B-Cell Lymphoma (DLBCL or DLBL)

Several DLBCL cell lines (SU-DHL-2, OCI-LY-3, TMD8, RI-1 (ABC subtype), and WSU-NHL (GCB subtype)) were plated in a 96-well plate at 5,000 cells per well and incubated overnight at 37° C. For each cell line, talimogene laherparepvec serially diluted (serial dilutions of 1:4) in nine wells, starting at 100 MOI. After a 72 hour incubation, the number of cells left in each well was quantified using CellTiter-Glo Luminescent cell viability assay (Promega, Madison, Wis.).

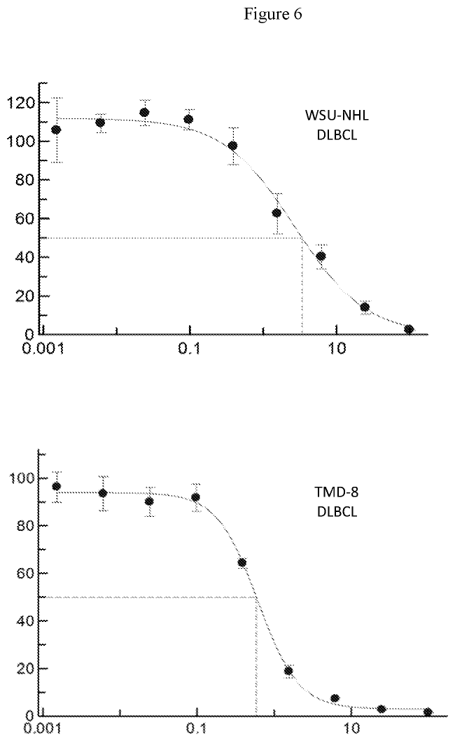

Talimogene laherparepvec was efficacious in 14 of 21 DLBCL cell lines at MOI below 100. Five cell lines (SU-DHL-2, OCI-LY-3, TMD8, RI-1 (ABC subtype), and WSU-NHL (GCB subtype)) demonstrated the most sensitivity with MOI IC₅₀ at or below 1 (Table 2). In contrast, OCI-LY-1, KARPAS422, WSU-DLCL2, SU-DHL-4, SU-DHL-10 and OCI-LY-7 (all of the GCB subtype), showed resistance to talimogene laherparepvec up to 100 MOI (Table 2). Inhibition of cell growth was maximal in most cells lines showing sensitivity below MOI IC₅₀ of 1. FIG. 6 shows the degree of cell growth inhibition achieved by increasing concentrations of talimogene laherparepvec in the WSU-NHL (GCB subtype) and TMD8 (ABC subtype) DLBCL cell lines. These results demonstrate that treating DLBCL cell lines with talimogene laherparepvec results in strong inhibition of DLBCL tumor cell growth.

TABLE 2
MOI IC₅₀ for 21 DLBCL cell lines representing
both the ABC and GCB subtypes.

| Cell Line | Indication | MOI IC₅₀ |
|---|---|---|
| DOHH2 | DLBCL | >100 |
| FARAGE | DLBCL | 16.769 |
| DB | DLBCL | 11.746 |
| SU-DHL-4 | DLBCL | >100 |
| VAL | DLBCL | 3.582 |
| RCK-8 | DLBCL | 6.058 |
| RI-1 | DLBCL | 1.053 |
| OCI-LY-7 | DLBCL | >100 |
| SU-DHL-10 | DLBCL | >100 |
| U2938 | DLBCL | 10.044 |
| WSU-DLCL2 | DLBCL | >100 |
| WSU-NHL | DLBCL | 0.520 |
| KARPAS 422 | DLBCL | >100 |
| SU-DHL-6 | DLBCL | 9.970 |
| OCI-LY-10 | DLBCL | 15.012 |
| TMD8 | DLBCL | 0.580 |
| OCI-LY-1 | DLBCL | >100 |
| OCI-LY-3 | DLBCL | 0.300 |
| OCI-LY-19 | DLBCL | 52.799 |
| SC-1 | DLBCL | 6.766 |
| SU-DHL-2 | DLBCL | 0.231 |

### Additional Solid Tumors

Various solid tumor cell lines (melanoma, non-small cell lung carcinoma, colorectal, head and neck squamous carcinoma, hepatocellular carcinoma, gastric carcinoma and triple negative breast carcinoma) were plated in a 96-well plate at 2,000-10,000 cell per well and incubated overnight at 37° C. For each cell line, talimogene laherparepvec serially diluted (serial dilutions of 1:4) in nine wells, starting at 100 MOI. After a 72 hour incubation, the number of cells left in each well was quantified using ATP-Lite (Perkin Elmer, Waltham, Mass.).

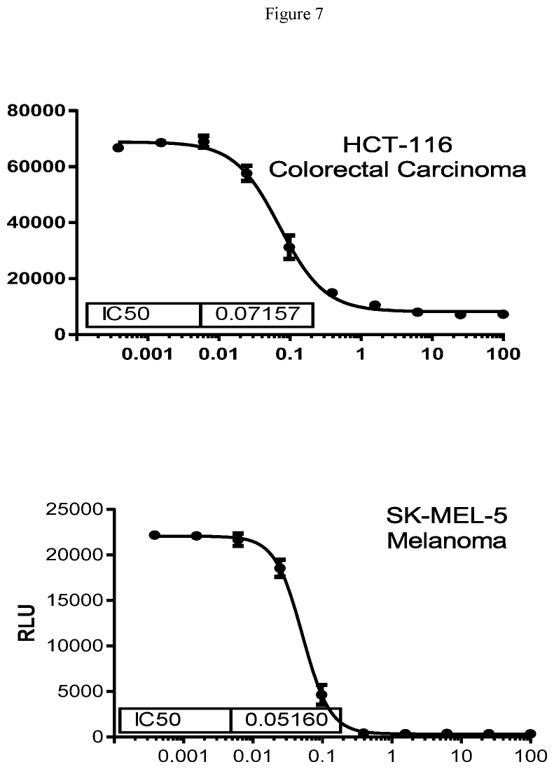

Talimogene laherparepvec was efficacious against all 13 melanoma and carcinoma cell lines tested. All cell lines tested showed MOI IC₅₀ below 1 (Table 3). FIG. 7 shows the degree of cell growth inhibition achieved by increasing concentrations of talimogene laherparepvec in the HCT-116 (colorectal) and SK-MEL-5 (melanoma) cell lines. These results demonstrate that treatment of melanoma, non-small cell lung carcinoma, colorectal, head and neck squamous carcinoma, hepatocellular carcinoma, gastric carcinoma and triple negative breast carcinoma cell lines with talimogene laherparepvec results in strong inhibition of tumor cell growth.

TABLE 3
MOI IC₅₀ for 13 cell lines representing
a variety of solid tumor indications.

| Cell Line | Indication | MOI IC₅₀ |
|---|---|---|
| SK-MEL-5 | Melanoma | 0.051 |
| M24met | Melanoma | 0.225 |
| A375 | Melanoma | 0.1 |
| A549 | NSCLC | 0.218 |
| SK-CO-1 | Colorectal | 0.145 |
| HT-29 | Colorectal | 0.135 |
| HCT-116 | Colorectal | 0.072 |
| FADU | HNSCC | 0.0133 |
| CAL 27 | HNSCC | 0.004 |
| SNU-182 | Hepatocellular | 0.03 |
| SNU-620 | Gastric | 0.162 |
| MDA-231 | Breast | 0.19 |
| Cal-51 | Breast | 0.56 |

### Cutaneous T-Cell Lymphoma (CTCL) and Multiple Myeloma (MM)

CTCL and MM cell lines were plated in a 96-well plate at 2,000-10,000 cell per well and incubated overnight at 37° C. For each cell line, talimogene laherparepvec serially diluted (serial dilutions of 1:4) in nine wells, starting at 100 MOI. After a 72 hour incubation, the number of cells left in each well was quantified using ATP-Lite (Perkin Elmer, Waltham, Mass.).

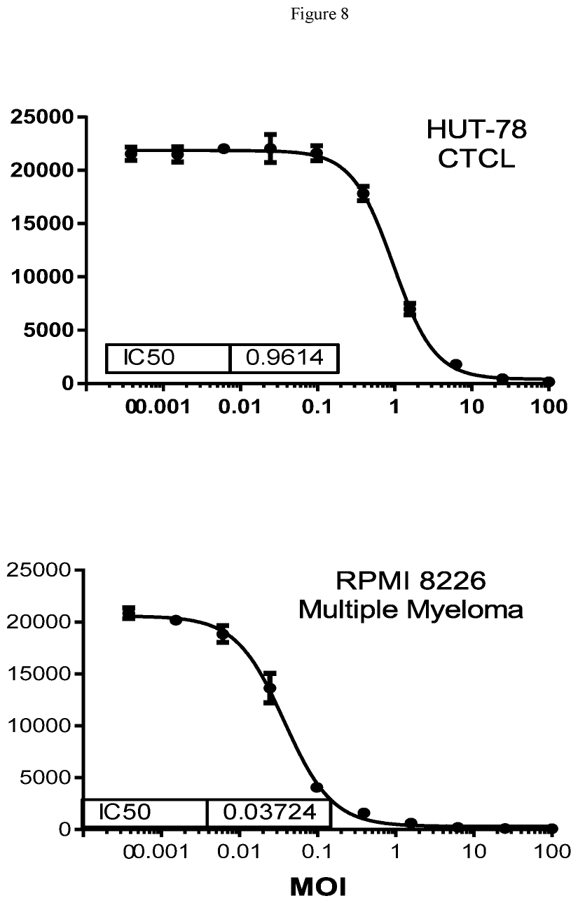

Talimogene laherparepvec was efficacious against all 5 cell lines tested (Table 4). Multiple myeloma cell lines showed higher sensitivity than the cutaneous T-cell lymphoma lines. FIG. 8 shows the degree of cell growth inhibition achieved by increasing concentrations of talimogene laherparepvec in the HUT-78 (CTCL) and RPMI 8226 (multiple myeloma) cell lines. These results demonstrate that treating cutaneous T-cell lymphoma (CTCL) and multiple myeloma (MM) cell lines with talimogene laherparepvec results in strong inhibition of tumor cell growth.

TABLE 4
MOI IC₅₀ for 2 cutaneous T-cell lymphoma
and three multiple myeloma cell lines.

| Cell Line | Indication | MOI IC₅₀ |
|---|---|---|
| HUT-78 | CTCL | 0.961 |
| HUT-102 | CTCL | 19.22 |
| KMS-12-BM | MM | 0.56 |
| RPMI8226 | MM | 0.037 |
| NCI-H929 | MM | 0.03 |

### Example 3

### Talimogene laherparepvec Inhibits the Growth of a Variety of Murine Tumor Cells Lines in Cell-Based Assays

Melanoma, colorectal carcinoma and B-cell lymphoma murine cell lines were plated in a 96-well plate at 2,000-10,000 cell per well and incubated overnight at 37° C. For each cell line, talimogene laherparepvec serially diluted (serial dilutions of 1:4) in nine wells, starting at 100 MOI. After a 72 hour incubation, the number of cells left in each well was quantified using ATP-Lite (Perkin Elmer, Waltham, Mass.).

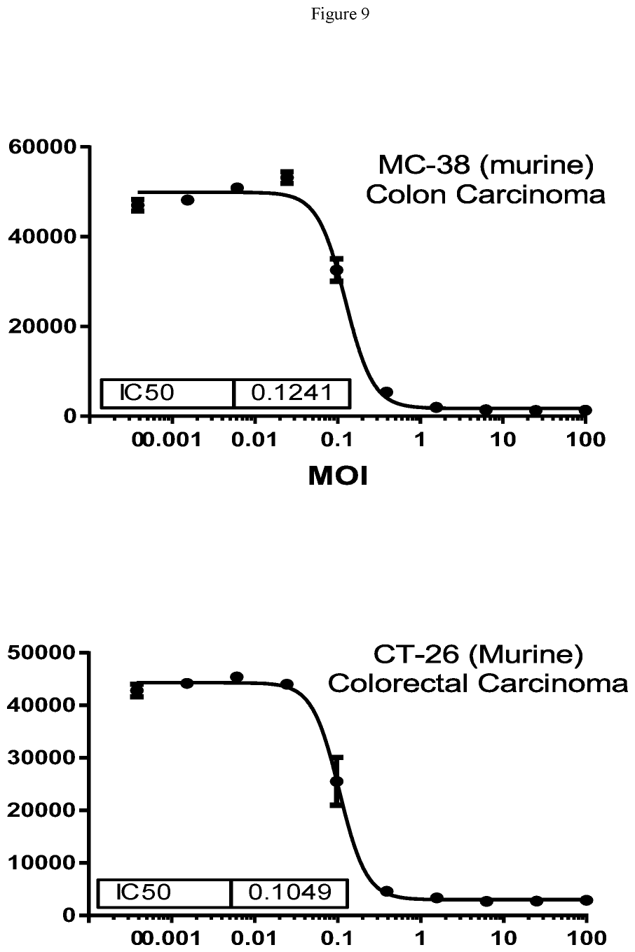

Talimogene laherparepvec was efficacious against 4 of 5 cell lines tested (Table 5). The B16F10 melanoma cell line demonstrated resistance to talimogene laherparepvec. This resistance is mediated by the lack of entry receptors for herpes simplex virus type 1 as previously described by Miller et al., Molecular Therapy, 3(2):160-168 (2001). The Cloudman CL M3 (melanoma), CT-26 and MC-38 (colorectal) cell lines displayed similar sensitivity with MOI IC₅₀ ˜0.2. FIG. 9 shows the degree of cell growth inhibition achieved by increasing concentrations of talimogene laherparepvec in the CT-26 and MC-38 (colorectal) cell lines. These results demonstrate that treating murine tumor cell lines (melanoma, colorectal carcinoma and B-cell lymphoma) with talimogene laherparepvec results in strong inhibition of tumor cell growth.

TABLE 5
MOI IC₅₀ for 5 murine cell lines.

| Cell Line | Indication | MOI IC₅₀ |
|---|---|---|
| Cloudman CL M3 | Melanoma | 0.17 |
| B16F10 | Melanoma | >100 |
| A20 | B-cell Lymphoma | 2.00 |
| CT-26 | Colorectal | 0.22 |
| MC-38 | Colorectal | 0.12 |

### Example 4

### OncoVex^{mGM-CSF} Inhibits the Growth of B-Cell Lymphoma and Neuroblastoma Tumors in a Mouse Model

A20 tumor cells were injected subcutaneously in the right and left flanks of female BALB/c mice (2×10⁶ cells) on day 0. Tumor volume (mm³) was measured using electronic calipers twice per week (Q2W). Once tumors reached an average of approximately 100 mm³, mice were randomized into groups (10 mice per group) such that the average tumor volume (in both flanks) and the variability of tumor volume at the beginning of treatment administration were uniform across treatment groups. Mice were then administered three intratumoral injections of OncoVex^{mGM-CSF} (3×10⁴-3×10⁶ PFU/dose) or vehicle on days 10, 13 and 16. Clinical signs, body weight changes, and survival (mice were removed from study when tumors reached 800 mm³) were measured 2-3 times weekly until study termination.

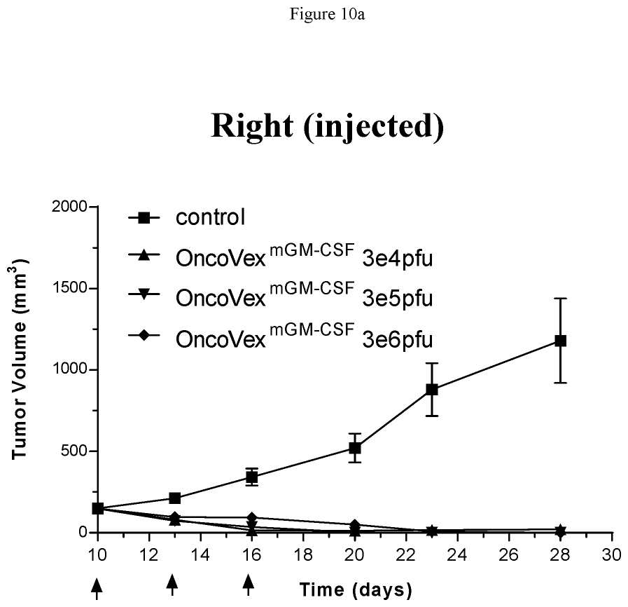

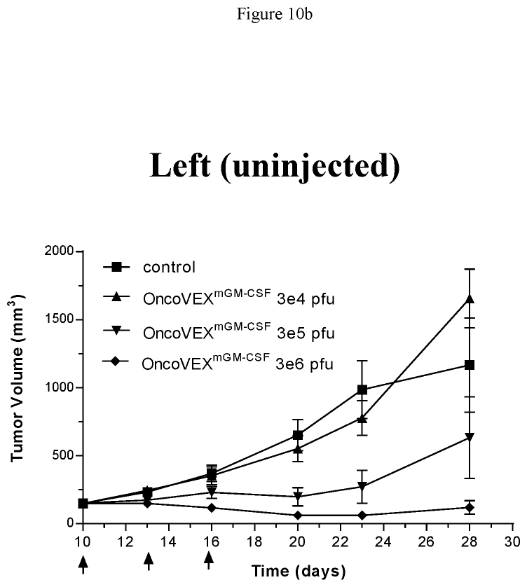

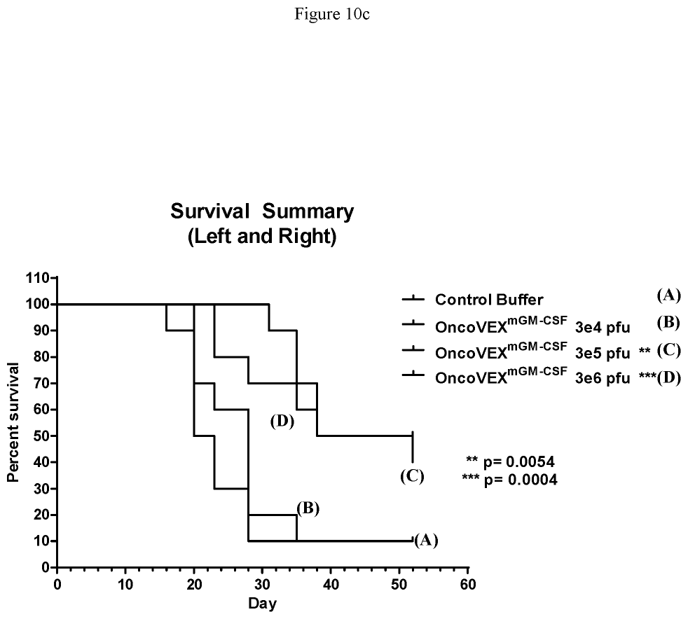

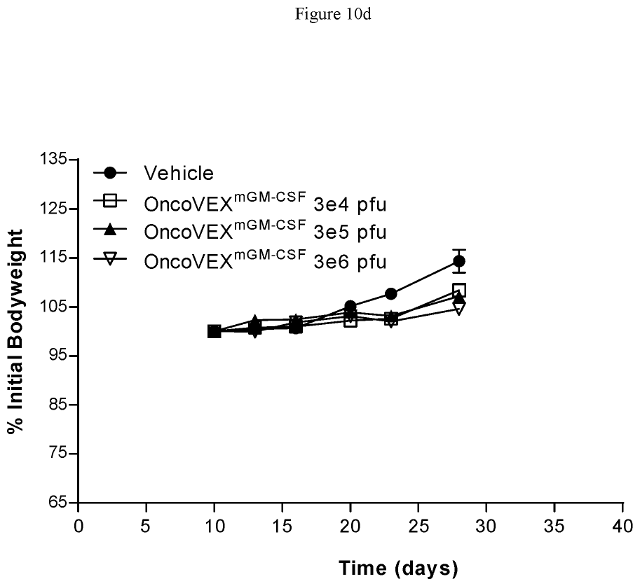

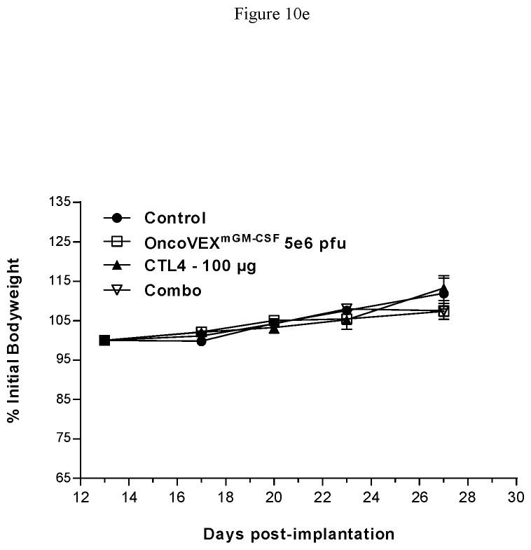

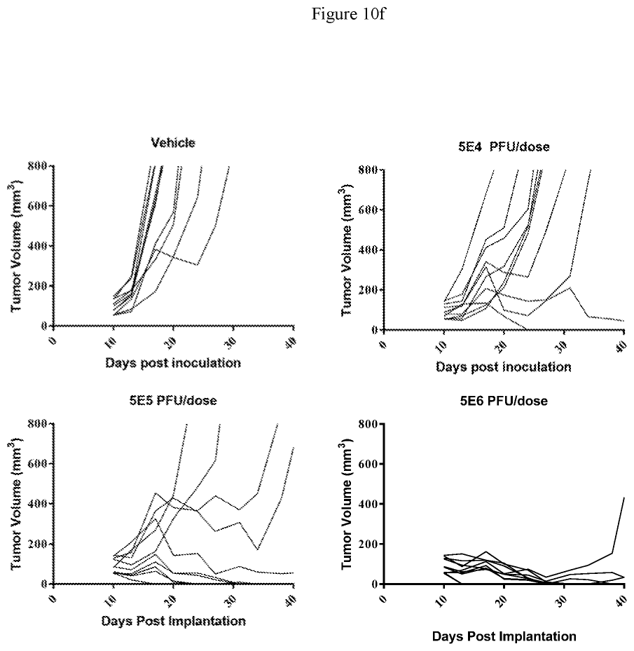

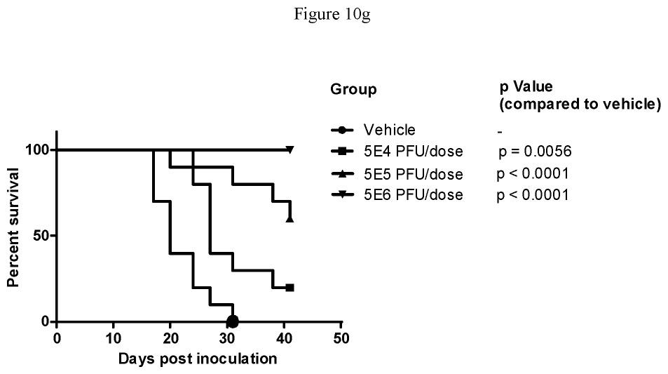

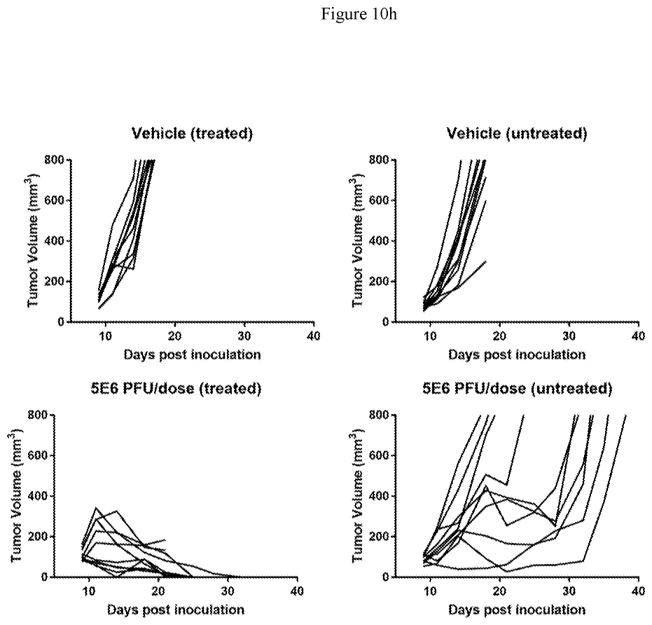

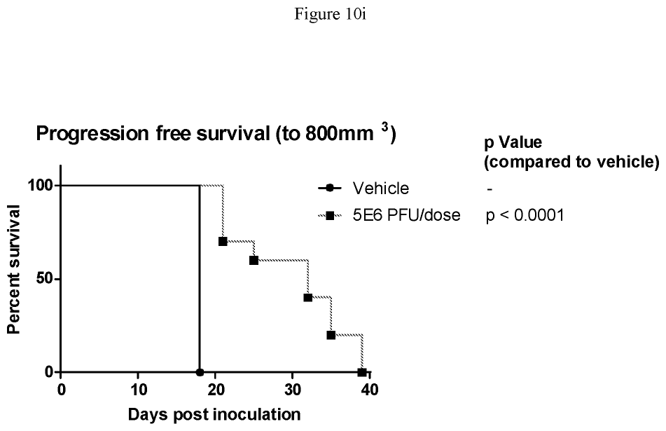

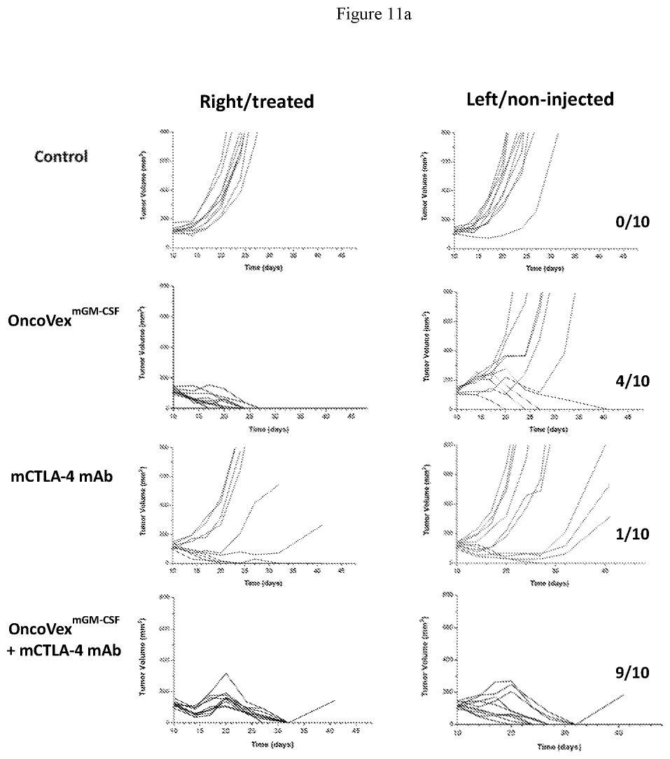

Treatment of A20 tumor bearing animals with OncoVex^{mGM-CSF} resulted in 100% complete regressions of all directly injected tumors at all three doses: 3×10⁴ PFU, 3×10⁵ PFU, and 3×10⁶ PFU (FIG. 10a). Contralateral tumors showed no response at the 3×10⁴ PFU dose, 50% tumor growth inhibition at the 3×10⁵ PFU dose, and 100% tumor growth inhibition at the 3×10⁶ PFU dose (FIG. 10b). Median survival was significantly increased in the 3×10⁵ PFU (p=0.0054) and 3×10⁶ PFU dose (p=0.0004) groups compared with vehicle (38 days vs. 21 days, respectively—FIG. 10c). No reduction in body weight were observed indicating that the treatments tested were safe and tolerable (FIGS. 10d and 10e). Table 6 shows the percent of subjects that were tumor-free in each group.

In addition, neuro2a neuroblastoma tumor-bearing mice were treated with OncoVex^{mGM-CSF} at 5×10⁴, 5×10⁵, or 5×10⁶ PFU/dose (n=10 per group). OncoVex^{mGM-CSF} was administered by intratumoral injection once daily on study days 10, 13 and 16 (FIG. 10f). Injected tumors were measured 2 times per week. Results are represented as individual tumor volume in mm³ as a function of time in days where day 0 is the day of tumor inoculation. Median survival was significantly increased in the 5×10⁴ PFU/dose (p=0.0056), 5×10⁵ PFU/dose (p<0.0001) and 5×10⁶ PFU/dose (p<0.0001) groups compared with vehicle (FIG. 10g).

The effect of OncoVex^{mGM-CSF} treatment on uninjected (“untreated”) tumors in neuro2a neuroblastoma tumor-bearing mice was also evaluated. Neuro2a tumor cells were implanted subcutaneously in the right and left flanks of female A/J mice (1×10⁶ cells) on day 0. Tumor volume (mm³) was measured using electronic calipers twice per week (Q2W). Once tumors reached an average of approximately 100 mm³, mice were randomized into groups (10 mice per group) such that the average tumor volume (in both flanks) and the variability of tumor volume at the beginning of treatment administration were uniform across treatment groups. Mice were then administered three intratumoral injections of OncoVEX^{mGM-CSF} (5×10⁶ PFU/dose) or vehicle on days 10, 13 and 16 on the right side (“treatment” side). Tumor volume and survival (mice were removed from study when tumors reached 800 mm³ on either side) were measured 2 times weekly until study termination (FIG. 10h). Treatment of Neuro2a tumor bearing animals with OncoVEX^{mGm-CSF} resulted in complete regressions in 8/10 directly injected tumors (FIG. 10h). Contralateral uninjected (“untreated”) tumors showed marked delay in tumor growth (FIG. 10h). Median survival was significantly increased in OncoVEX^{mGM-CSF}-treated groups compared with vehicle (32 days vs. 18 days, p<0.0001, FIG. 10i).

These results demonstrate that treatment of established B-cell lymphoma and neuroblastoma tumors with OncoVex^{mGM-CSF} in an in vivo mouse model results in strong inhibition of tumor growth. Anti-tumor activity was observed in directly injected tumors (presumably via oncolysis and immune response) and in non-injected contralateral tumors in the same host (presumably via adaptive immune response).

TABLE 6

| | | | SUBJECTS | SUBJECTS |
|---|---|---|---|---|
| | MEDIAN | | TUMOR- | TUMOR- |
| | SUR- | SUBJECTS | FREE | FREE |
| GROUP | VIVAL | CENSORED | Injected | Contralateral |
| Vehicle | 21 | 0 | 1/10 | 1/10 |
| OncoVex^{mGM-CSF} | 28 | 0 | 8/10 | 0/10 |
| 3 × 10⁴ PFU | | | | |
| OncoVex^{mGM-CSF} | 38 | 0 | 9/10 | 5/10 |
| 3 × 10⁵ PFU | | | | |
| OncoVex^{mGM-CSF} | 38 | 0 | 10/10 | 5/10 |
| 3 × 10⁶ PFU | | | | |

### Example 5

### A Combination of OncoVex^{mGM-CSF} and CTLA-4 or PD-L1 Blockade Inhibits B-Cell Lymphoma Tumor Growth in a Mouse Model

A20 tumor cells were injected subcutaneously in the right and left flanks of female BALB/c mice (2×10⁶ cells) on day 0. Tumor volume (mm³) was measured using electronic calipers twice per week (Q2W). Once tumors reached an average of approximately 100 mm³, animals were randomized into groups (10 mice per group) such that the average tumor volume (in both flanks) and the variability of tumor volume at the beginning of treatment administration were uniform across treatment groups. Animals were then administered three intratumoral injections of OncoVex^{mGM-CSF} (5×10⁶ PFU/dose) alone or in combination with intraperitoneal injections of anti-PD-L1 mAb or anti-CTLA-4 mAb. Clinical signs, body weight changes, and survival (mice were removed from study when tumors reached 800 mm³) were measured 2-3 times weekly until study termination.

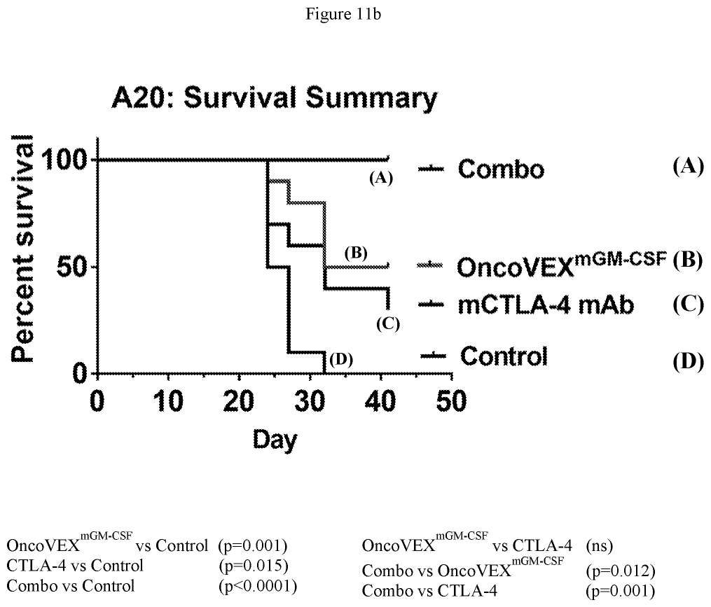

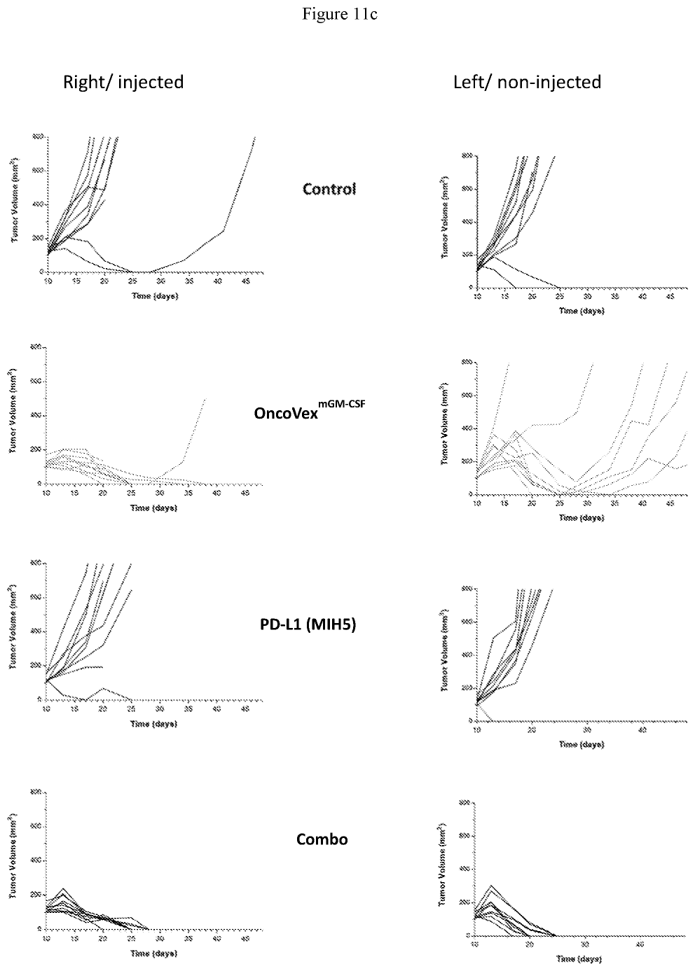

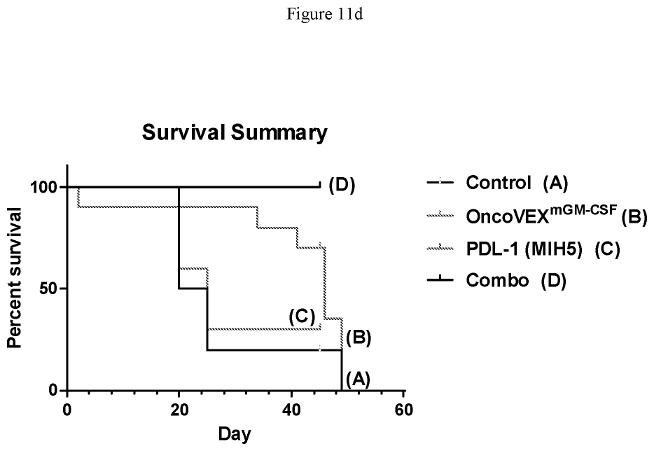

Treatment of A20 tumor bearing animals with OncoVex^{mGM-CSF}, an anti-CTLA-4 mAb, or the combination of OncoVex^{mGM-CSF} with an anti-CTLA-4 mAb, resulted in complete regression of all directly injected tumors (FIG. 11a). Tumors treated with intraperitoneal anti-CTLA-4 only (both flank) showed a 50% inhibition of tumor growth. Contralateral tumors showed some consistent effects with OncoVex^{mGM-CSF} alone, while the combination of OncoVex^{mGM-CSF} and anti-CTLA-4 mAb regressed all tumors and led to complete cures in 9/10 mice. Median survival was significantly increased in the anti CTLA-4 mAb group, the OncoVex^{mGM-CSF} group, and the combination of anti CTLA-4 mAb and OncoVex^{mGM-CSF} group vs. vehicle. In addition, a significant increase in median survival was measured in the combination group vs. either single agent alone (p=0.012 vs OncoVex^{mGM-CSF}, p=0.001 vs anti-CTLA-4 mAb). Median survival was 25.5 days for vehicle, 36.5 days for the OncoVex^{mGM-CSF} group, and 32 for the anti CTLA-4 mAb group. The median survival for the combination group remained undefined past day 40 at which time 9 out of 10 mice showed no signs of tumor. (FIG. 11b and Table 7a).

TABLE 7a

| | | SUBJECTS | SUBJECTS |
|---|---|---|---|
| | | TUMOR- | TUMOR- |
| | MEDIAN | FREE | FREE |
| GROUP | SURVIVAL | Injected | Contralateral |
| Control | 25.5 | 0/10 | 0/10 |
| OncoVex^{mGM-CSF} | 36.5 | 10/10 | 4/10 |
| 5 × 10⁶ PFU | | | |
| mCTLA-4 mAb | 32 | 4/10 | 1/10 |
| 30 μg | | | |
| OncoVex^{mGM-CSF} + | undefined | 9/10 | 9/10 |
| mCTLA-4 mAb | | | |

Treatment of A20 tumor bearing animals with OncoVex^{mGM-CSF}, an anti-PD-L1 mAb, or the combination of OncoVex^{mGM-CSF} with an anti-PD-L1 mAb, resulted in complete regression of all directly injected tumors (FIG. 11c). Tumors treated with intraperitoneal anti-PD-L1 mAb only (both flank) showed no effect on tumor growth. Contralateral tumors showed some consistent effects with OncoVex^{mGm-CSF} alone, while the combination of OncoVex^{mGm-CSF} and anti-PD-L1 mAb regressed all tumors and led to complete cures in 10/10 mice. Median survival was significantly increased in the OncoVex^{mGM-CSF} and combination groups vs. vehicle. In addition, a significant increase in median survival was measured in the combination group vs. anti-PD-L1 mAb alone. A strong trend in overall survival was also observed when the combination was compared to OncoVex^{mGM-CSF} although statistical significance was not observed (p=0.067 vs OncoVex^{mGM-CSF}, p=0.0013 vs anti-PD-L1 mAb). Median survival was 23 days for vehicle, 48 days for the OncoVex^{mGM-CSF} group, and 26 days for the anti-PD-L1 mAb group. The median survival for the combination group remained undefined past day 40 at which time 10 out of 10 mice showed no signs of tumor. (FIG. 11d and Table 7b).

TABLE 7b

| | MEDI- | | SUBJECTS | SUBJECTS |
|---|---|---|---|---|
| | AN | | TUMOR- | TUMOR- |
| | SUR- | SUBJECTS | FREE | FREE |
| GROUP | VIVAL | CENSORED | Injected | Non-injected |
| Vehicle | 22.5 | 0 | 1 | 2 |
| OncoVex^{mGM}^{_{—}}^{CSF} | 46 | 0 | 6 | 3 |
| 3 × 10⁵ PFU | | | | |
| PD-L1 (MIH5) | 25 | 1* | 1 | 1 |
| Combo | unde- | 0 | 10 | 10 |
| | fined | | | |

These results demonstrate that treating established B-cell lymphoma tumors in an in vivo mouse model with OncoVex^{mGM-CSF} in combination with either PD-L1 or CTLA-4 blockade results in better inhibition of tumor growth compared to either single agent alone. Anti-tumor activity was observed in directly injected tumors (presumably via oncolysis and immune response) and in non-injected contralateral tumors in the same host (presumably via adaptive immune response).

### Example 6

### OncoVex^{mGM-CSF}, Either Alone or in Combination with CTLA-4 or PD-L1 Blockade, Inhibits the Growth of Colorectal Tumors in a Mouse Model

CT-26 tumor cells were injected subcutaneously in the right and left flanks of female BALB/c mice (2×10⁶ cells) on day 0. Tumor volume (mm³) was measured using electronic calipers twice per week (Q2W). Once tumors reached an average of approximately 100 mm³, animals were randomized into 4 groups (10 mice per group) such that the average tumor volume (in both flanks) and the variability of tumor volume at the beginning of treatment administration were uniform across treatment groups. Animals were then administered: a) PBS+IgG control; b) OncoVex^{mGM-CSF}+IgG control; c) PBS+anti CTLA-4 mAb, or PBS+anti PD-L1 mAb; or d) OncoVex^{mGM-CSF}+anti CTLA-4 mAb, or OncoVex^{mGM-CSF}+anti PD-L1 mAb. Clinical signs, body weight changes, and survival (mice were removed from study when tumors reached 800 mm³) were measured 2-3 times weekly until study termination.

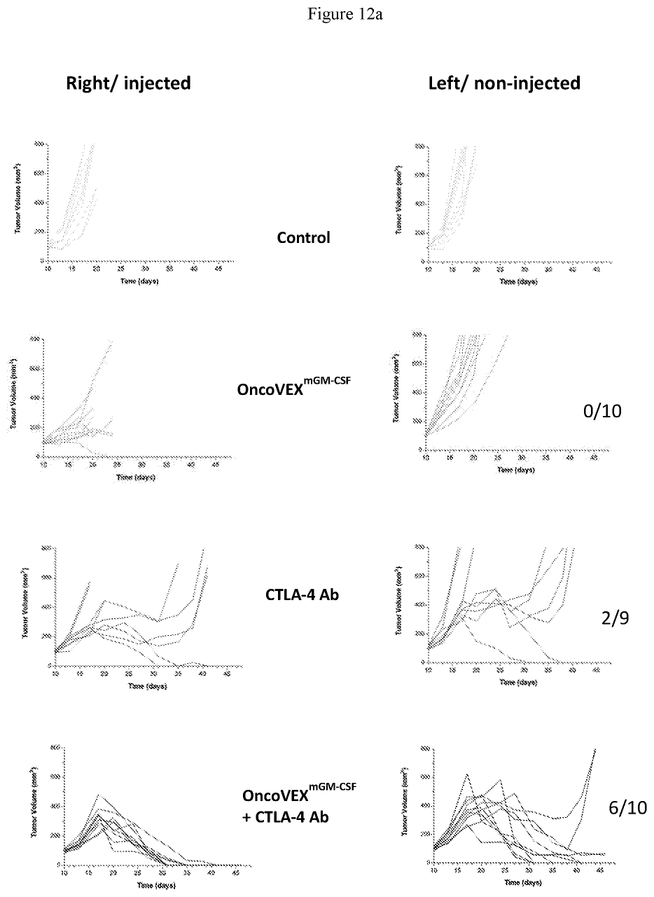

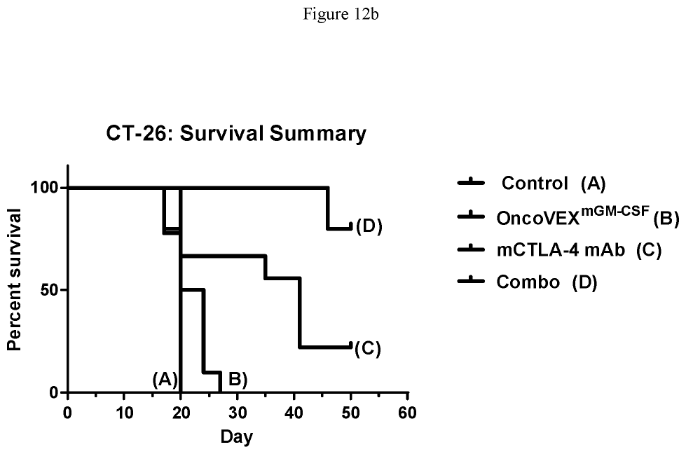

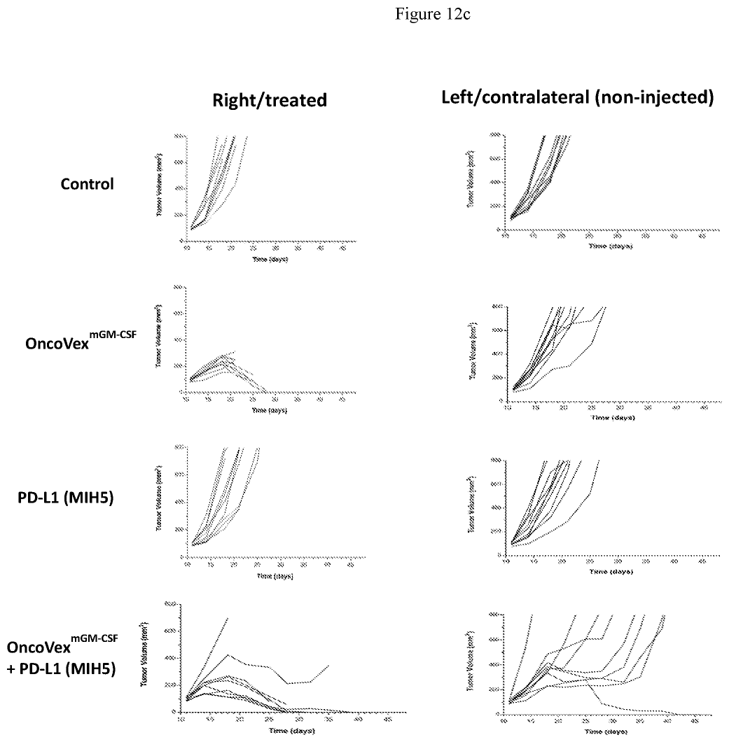

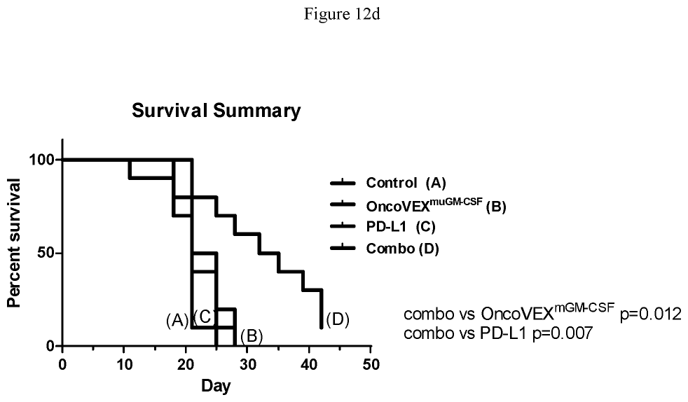

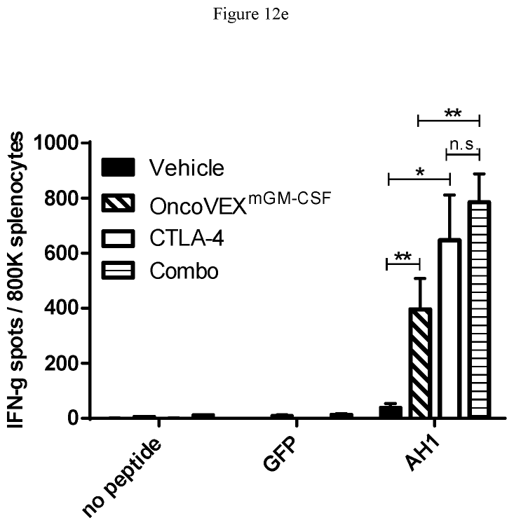

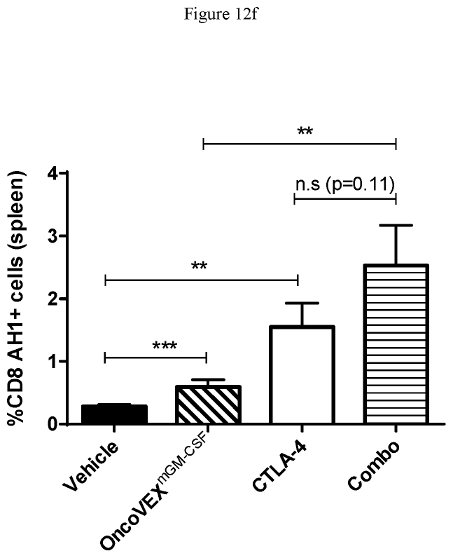

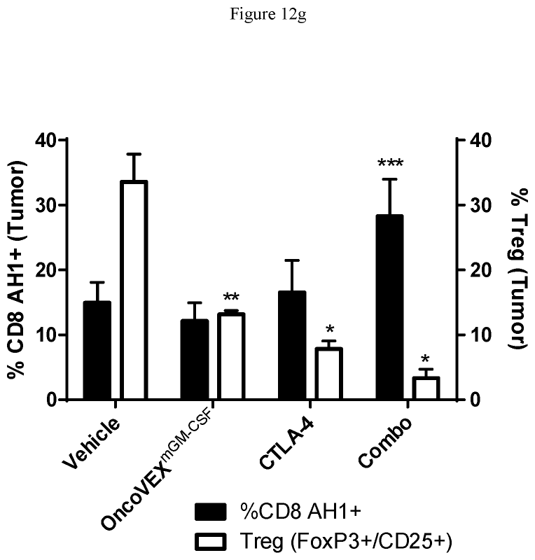

Treatment of CT-26 tumor bearing animals with OncoVex^{mGM-CSF}, anti CTLA-4 mAb, or a combination thereof resulted in 75% tumor growth inhibition of all directly injected tumors by day 20 (data not shown). Contralateral tumors showed no response to OncoVex^{mGm-CSF} alone (˜25% TGI), while ˜75% tumor growth inhibition was observed in the anti CTLA-4 mAb and combination groups. Median survival was significantly increased in the anti CTLA-4 mAb and combination groups vs. vehicle (FIG. 12b). Median survival was 20 days for vehicle, 22 days for OncoVEX^{mGm-CSF} and 41 days for the anti CTLA-4 group. The median survival for the combination was longer than 50 days and remained undefined when the experiment was stopped. Median survival in the combination groups was significantly longer than in either OncoVex^{mGM-CSF} (p=0.0001) or anti CTLA-4 mAb (p=0.0031) groups alone. (FIG. 12b and Table 8a).

TABLE 8a

| | | SUBJECTS | SUBJECTS |
|---|---|---|---|
| | | TUMOR- | TUMOR- |
| | MEDIAN | FREE | FREE |
| GROUP | SURVIVAL | Injected | Contralateral |
| Control | 20 | 0/10 | 0/10 |
| OncoVex^{mGM-CSF} | 22 | 1/10 | 0/10 |
| 5 × 10⁶ PFU | | | |
| mCTLA-4 mAb | 41 | 2/9 | 2/9 |
| 30 μg | | | |
| OncoVex^{mGM-CSF} + | undefined | 10/10 | 6/10 |
| mCTLA-4 mAb | | | |

Treatment of CT-26 tumor bearing animals with OncoVex^{mGM-CSF} or the combination of OncoVex^{mGM-CSF} and anti-PD-L1 mAb resulted in 75% tumor growth inhibition of all directly injected tumors by day 18 (data not shown). Intraperitoneal injection with anti-PD-L1 mAb had little effect on tumor growth (either flank). Contralateral tumors showed no response to OncoVex^{mGM-CSF} or anti PD-L1 mAb alone while 75% tumor growth inhibition was observed in the combination group (FIG. 12c). Median survival was significantly increased in the combination group vs. either agent alone (p=0.012 vs OncoVex^{mGM-CSF}, p=0.007 vs anti-PD-L1 mAb). Median survival was 21 days for vehicle and anti PD-L1 mAb groups, 23 days for OncoVex^{mGM-CSF}, and 34 days for the combination group. (FIG. 12d and Table 8b).

TABLE 8b

| | | SUBJECTS | SUBJECTS |
|---|---|---|---|
| | | TUMOR- | TUMOR- |
| | MEDIAN | FREE | FREE |
| GROUP | SURVIVAL | Injected | Contralateral |
| Control | 21 | 0 | 0 |
| OncoVex^{mGM-CSF} | 23 | 1 | 0 |
| 5 × 10⁶ PFU | | | |
| PD-L1 (MIH5) | 21 | 0 | 0 |
| 300 μg | | | |
| OncoVex^{mGM-CSF} + | 34 | 5 | 1 |
| PD-L1 (MIH5) | | | |

These results demonstrate that treating established colorectal tumors in an in vivo mouse model with: 1) OncoVex^{mGM-CSF}; 2) a combination of OncoVex^{mGM-CSF} and anti-CTLA-4 blockage; or 3) a combination of OncoVex^{mGM-CSF} and anti-PD-L1 blockade, results in strong inhibition of tumor growth. Anti-tumor activity was observed in directly injected tumors (presumably via oncolysis and immune response) with: 1) OncoVex^{mGM-CSF}; 2) a combination of OncoVex^{mGM-CSF} and anti-CTLA-4 blockage; and 3) a combination of OncoVex^{mGM-CSF} and anti-PD-L1 blockade. Anti-tumor activity was observed in non-injected contralateral tumors in the same host (presumably via adaptive immune response) with: 1) a combination of OncoVex^{mGM-CSF} and anti-CTLA-4 blockage; and 2) a combination of OncoVex^{mGM-CSF} and anti-PD-L1 blockade.

In order to further understand the anti-tumor activity of the combination of OncoVex^{mGM-CSF} and anti-CTLA-4 blockage, the ability of: 1) OncoVex^{mGM-CSF}; 2) anti-CTLA-4 blockage; and 3) a combination of OncoVex^{mGM-CSF} and anti-CTLA-4 blockage to release tumor antigens and stimulate anti-tumor specific T-cell responses was evaluated. 96-well ELISpot plates with a nitrocellulose filter base (Millititer HA; Millipore, Temecula, Calif.) were coated with purified anti-IFN-γ (2 μg/ml) antibody. Splenocytes (8×10⁵) from OncoVex^{mGM-CSF}, anti-CTLA-4 mAb, or the combination-treated CT-26 tumor-bearing mice on day 10 were incubated with control peptides (GFP) or the AH1 peptide (SPSYVHQF) at a final concentration of 1 μM for 20 hours at 37° degrees. The AH1 peptide is an immunodominant Ag derived from the envelope protein (gp70) of the endogenous murine leukemia virus presented by the MHC class I L^{d} molecule (25). Spots were enumerated using a CTLS6 Fluorospot analyzer (CTL, Shaker Heights, Ohio). Quantification of systemic (splenic) anti-AH1 CD8⁺ T-cells by ELISpot or by dextramer staining using FACS demonstrated a significant increase in AH1 reactive T-cells in mice treated with OncoVex^{mGM-CSF}, CTLA-4 blockade, or the combination of OncoVex^{mGM-CSF} and CTLA-4 blockade (FIGS. 12e, 12f). Quantification of local (tumor) anti-AH1 CD8⁺ T-cells showed a significant increase only in the combination group. A significant decrease in Tregs was also observed in the CT-26 model. This effect was greater in combination with CTLA-4 blockade (FIG. 12g). This experiment demonstrates that the combination of OncoVex^{mGM-CSF} and anti-CTLA-4 blockage leads to an increase in the presence of effector cells and a decrease in the presence of regulatory T cells, which in turn leads to an increase in efficacy of the combination over either compound alone.

### Example 7

### OncoVex^{mGM-CSF}, Either Alone or in Combination with CTLA-4 Blockade, Inhibits the Growth of Melanoma Tumors in a Mouse Model

B16F10 cells (5×10⁴resistant to OncoVex^{mGm-CSF} lysis due to a lack of an HSV-1 entry receptor) were injected intravenously on day 0. On day 2, B16F10 melanoma cells transfected with mouse Nectin 1 (sensitive to OncoVex^{mGM-CSF} lysis) were injected subcutaneously in the right flank of female BL6 mice. Tumor volume (mm³) was measured using electronic calipers twice per week (Q2W). Once subcutaneous tumors reached an average of approximately 100 mm³, animals were randomized into four groups (10 mice per group) such that the average tumor volume and the variability of tumor volume at the beginning of treatment administration were uniform across treatment groups. Animals were then administered three intratumoral injections of OncoVex^{mGM-CSF} (5×10⁶ PFU/dose), four intraperitoneal injections of anti-CTLA-4 mAb, a combination of three intratumoral injections of OncoVex^{mGM-CSF} (5×106 PFU/dose) with four intraperitoneal injections of anti-CTLA-4 mAb, or vehicle alone. Clinical signs, body weight changes, and survival (mice were removed from study when tumors reached 800 mm³) were measured 2-3 times weekly until study termination.

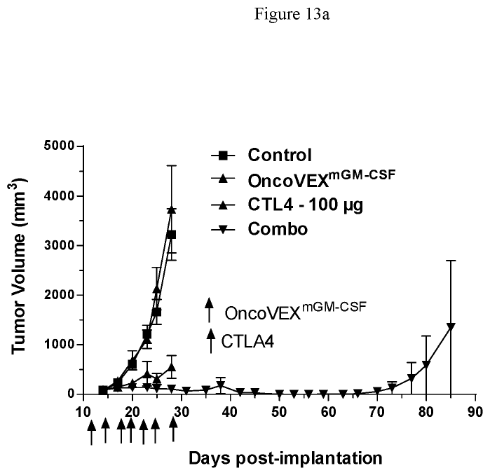

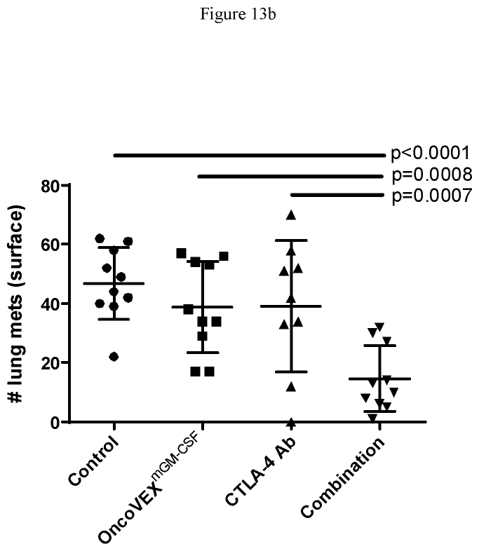

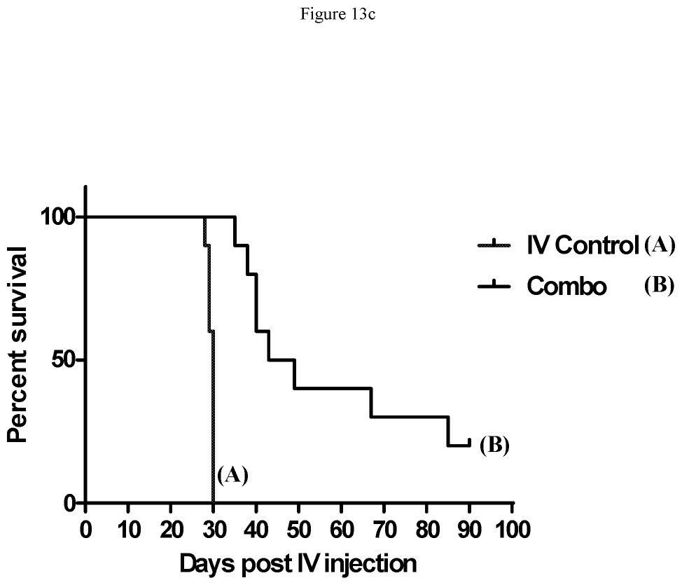

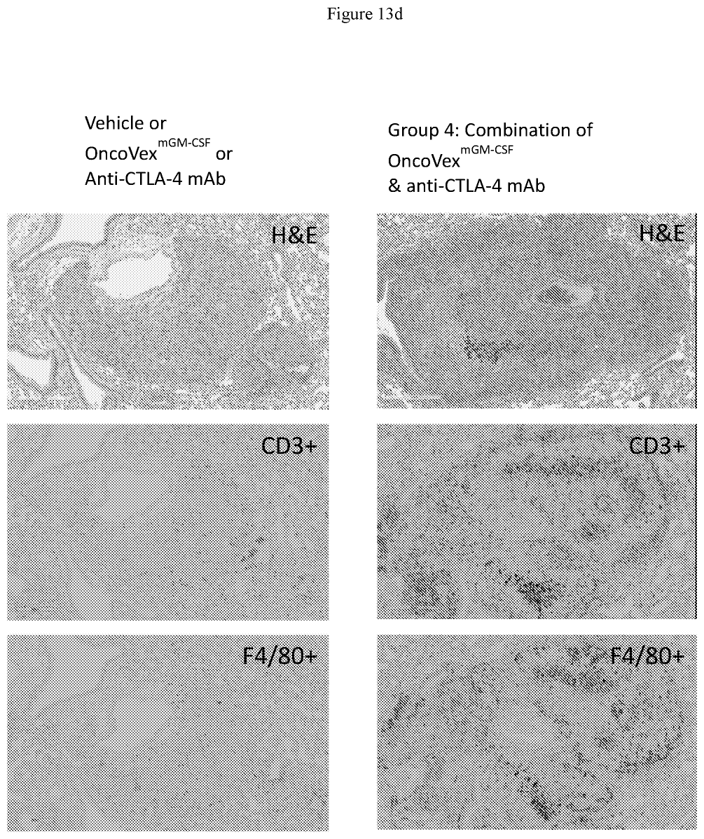

Treatment of B16F10 Nectin 1 tumor bearing mice with OncoVex^{mGM-CSF}, or the combination of OncoVex^{mGM-CSF} and anti CTLA-4 mAb resulted in 85% and 99% tumor (subcutaneous) growth inhibition respectively. No inhibition was observed with anti CTLA-4 mAb alone (FIG. 13a). Assessment of lung metastasis burden showed that the combination of OncoVex^{mGM-CSF} and anti CTLA-4 mAb was significantly more effective than either treatment alone (p=0.0008 vs OncoVex^{mGM-CSF}, p=0.0007 vs anti CTLA-4 mAb) in inhibiting lung metastasis (FIG. 13b). Median survival was significantly increased in the combination group vs. vehicle (p<0.0001). Median survival was 30 days for vehicle and 46 days for OncoVex^{mGm-CSF} plus anti CTLA-4 mAb group (FIG. 13c and Table 9).

TABLE 9

| Group | | | mOS |
|---|---|---|---|
| (n = 10) | Treatment | Tumors | (days) |
| 1 | IV Control | Lung only | 30 |
| 2 | OncoVex^{mGM-CSF} + | SC and lung | 46 |
| | CTLA-4 | | |

These results demonstrate that treating established melanoma tumors in an in vivo mouse model with OncoVex^{mGM-CSF}, or the combination of OncoVex^{mGM-CSF} and anti CTLA-4 mAb results in strong inhibition of tumor growth. These results also show that the combination of OncoVex^{mGM-CSF} and anti CTLA-4 mAb demonstrates a significantly more robust systemic anti-tumor effect, compared to either treatment alone, as evidenced by the ability of the combination to inhibit lung metastases. Anti-tumor activity was observed in directly injected tumors (presumably via oncolysis and immune response) and in non-injected contralateral tumors in the same host (presumably via adaptive immune response).

It was also observed that lung tumors in the vehicle, OncoVex^{mGM-CSF}, and anti-CTLA-4 mAb groups showed rare to scattered T and B cells at the tumor periphery and mild intratumoral macrophages. Interestingly, tumors in the OncoVex^{mGM-CSF} and anti-CTLA-4 mAb combination group showed prominent T cells in the tumor periphery as well as T cell infiltration into the tumor in variable numbers. Macrophages were prominent both in the tumor and in dense cellular infiltrates at the tumor periphery. B cells remained exclusively at the tumor periphery (FIG. 13d and data not shown).

These lung tumor immune infiltrate results indicate that the combination of OncoVex^{mGM-CSF} and CTLA-4 blockade can convert converted poorly infiltrated B16F10 metastases (i.e., “cold” tumors) into well infiltrated B16F10 metastasis (i.e., “hot” tumors).

### Example 8

### OncoVex^{mGM-CSF} Inhibits the Growth of Triple Negative Breast Carcinoma Tumors in a Mouse Model

4T1 tumor cells were injected subcutaneously in the right flanks of female BALB/c mice (2×10⁶ cells) on day 0. Tumor volume (mm³) was measured using electronic calipers twice per week (Q2W). Once tumors reached an average of approximately 100 mm³, animals were randomized into four groups (10 mice per group) such that the average tumor volume and the variability of tumor volume at the beginning of treatment administration were uniform across treatment groups. Animals were then administered three intratumoral injections of OncoVex^{mGM-CSF} (5×10⁴, 5×10⁵, or 5×10⁶ PFU/dose) or vehicle. Clinical signs, body weight changes, and survival (mice were removed from study when tumors reached 800 mm³) were measured 2-3 times weekly until study termination.

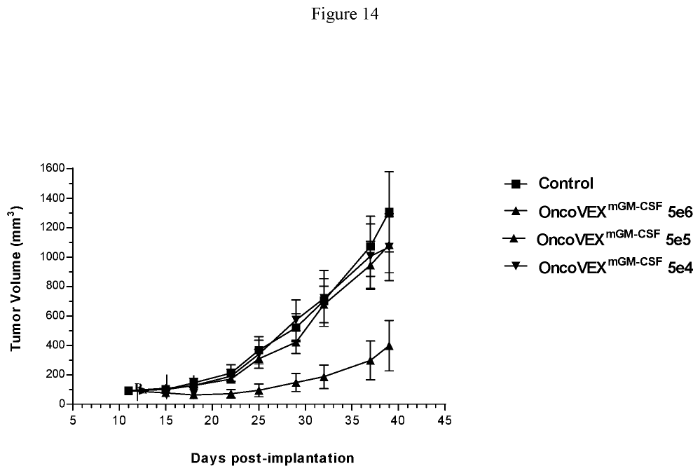

Treatment of 4T1 tumor bearing animals with OncoVex^{mGM-CSF} resulted in 75% tumor growth inhibition at the 5×10⁶ PFU/dose group (p<0.0001). The 5×10⁴ and 5×10⁵ doses of OncoVex^{mGM-CSF} did not result in any measurable tumor growth inhibition (FIG. 14).

These results demonstrate that treating established triple negative breast carcinoma tumors with OncoVex^{mGM-CSF} results in strong inhibition of tumor growth. Anti-tumor activity was measured in directly injected tumors only.

### Example 9

### OncoVex^{mGM-CSF}, Either Alone or in Combination with a GITR Agonist, Inhibits the Growth of B-Cell Lymphoma Tumors in a Mouse Model

A20 tumor cells were injected subcutaneously in the right and left flanks of female BALB/c mice (2×10⁶ cells) on day 0. Tumor volume (mm³) was measured using electronic calipers twice per week (Q2W). Once tumors reached an average of approximately 100 mm³, animals were randomized into 4 groups (10 mice per group) such that the average tumor volume (in both flanks) and the variability of tumor volume at the beginning of treatment administration were uniform across treatment groups. Animals were then administered OncoVex^{mGM-CSF} or a combination of OncoVex^{mGM-CSF} and anti GITR mAb. Clinical signs, body weight changes, and survival (mice were removed from study when tumors reached 800 mm³) were measured 2-3 times weekly until study termination.

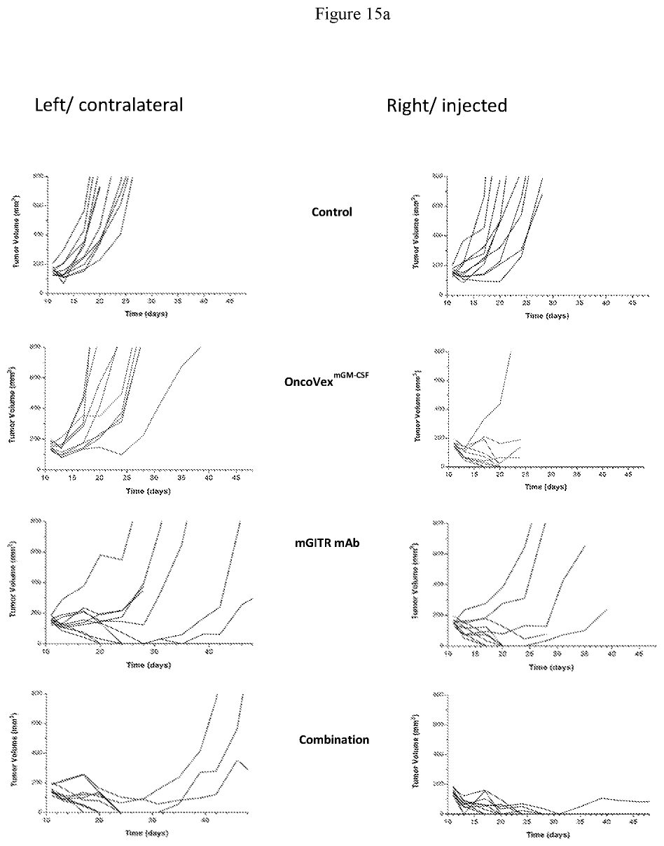

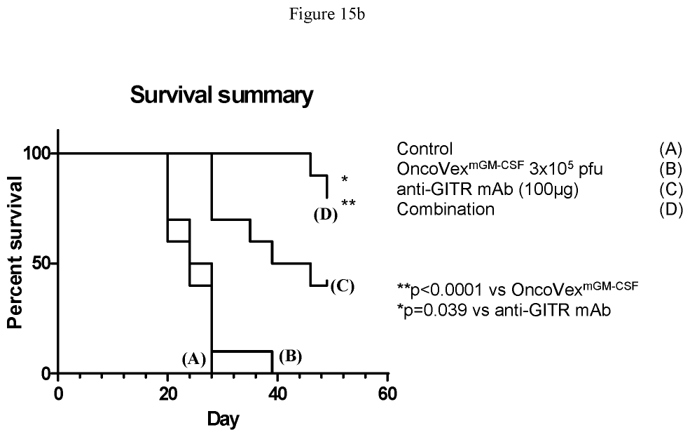

Tumors treated with intraperitoneal anti-GITR mAb cured 30% of tumors (both injected and contralateral). OncoVex^{mGM-CSF} cured 6/10 (60%) of injected tumors, whereas contralateral tumors showed a modest response with no cures. The combination of OncoVex^{mGM-CSF} and anti-GITR mAb regressed all tumors (both injected and contralateral) and led to complete cures in 7/10 mice. See FIG. 15a. Median survival was significantly increased in the anti GITR mAb group, the OncoVex^{mGM-CSF} group, and the combination of OncoVex^{mGM-CSF} group and anti GITR mAb, vs. vehicle. In addition, a significant increase in median survival was measured in the OncoVex^{mGM-CSF} group and anti-GITR mAb combination group vs. either single agent alone (p<0.0001 vs OncoVex^{mGM-CSF}, p=0.039 vs anti-GITR mAb). Median survival was 24 days for vehicle, 26 days for the OncoVex^{mGM-CSF} group, and 43 for the anti GITR mAb group. The median survival for the combination group remained undefined past day 49 at which time 7 out of 10 mice showed no signs of tumor. See FIG. 15b and Table 10.

TABLE 10

| | | SUBJECTS | SUBJECTS |
|---|---|---|---|
| | | TUMOR- | TUMOR- |
| | MEDIAN | FREE | FREE |
| Group | SURVIVAL | Injected | Contralateral |
| Control | 24 | 0/10 | 0/10 |
| OncoVex^{mGM-CSF} | 26 | 6/10 | 0/10 |
| 3 × 10⁵ PFU | | | |
| mGITR mAb | 43 | 4/10 | 3/10 |
| 100 ug | | | |
| OncoVex^{mGM-CSF} + | undefined | 8/10 | 7/10 |
| mGITR mAb | | | |

These results demonstrate that treating B-cell lymphoma tumors with OncoVex^{mGM-CSF} and a combination of OncoVex^{mGM-CSF} group and anti GITR mAb results in strong inhibition of tumor growth.

### Example 10

### Study Evaluating the Combination of OncoVex^{muGM-CSF} with PD-1 Inhibition in a Mouse Colon (Colorectal) Adenocarcinoma (MC-38) Tumor Model

This study was designed to evaluate the tolerability and anti-tumor activity of OncoVex^{muGM-CSF}, PD-1 inhibition, or the combination of OncoVex^{muGM-CSF} and PD-1 inhibition in a mouse MC-38 tumor model.

C57BL76 mice were inoculated with syngeneic MC-38 tumor cells on both the right and left flanks. Ten days after inoculation tumors averaged 5 mm in diameter (60 mm³ tumor volume) and animals were allocated into 6 groups (n=10/group). OncoVEX^{muGM-CSF} (5×10⁶ PFU/dose) or formulation buffer control were dosed intratumorally into the right side tumors once daily even, three days for three total doses. The left side tumors received no injection.

Anti-mouse PD-1 (clone RMP1-14, BioXCell) or isotype control antibody (rat IgG2a, clone 2A3, BioXCell) were dosed intraperitoneally at either 1 mg/dose or 300 μg/dose twice weekly beginning on study day 10 and ending on study day 30 (7 doses given). Tumor volumes of both the injected (right side) tumors and the non-injected (left side) tumors, body weights and gross clinical observations were collected 2-3 times weekly. Animals were euthanized if the total tumor volume (right+left) reached the IACUC mandated cut-off of >10% of body weight or if animals exhibited signs of distress. Peripheral blood was drawn on study days 14 and 20 (4 and 10 days, respectively, after the start of dosing) for immunophenotyping analysis. After red blood cell lysis, the white cells were stained for the following markers: CD3, CD4, CD8, CD25, CD49b (NK marker), FoxP3, GITR, PD-1 and PD-L1 and analyzed by flow cytometry.

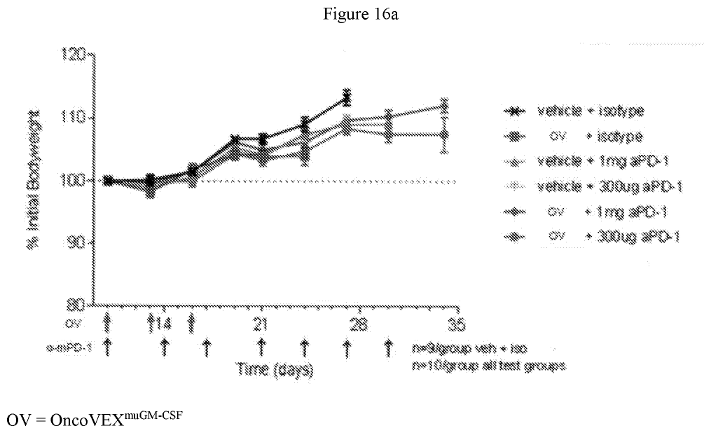

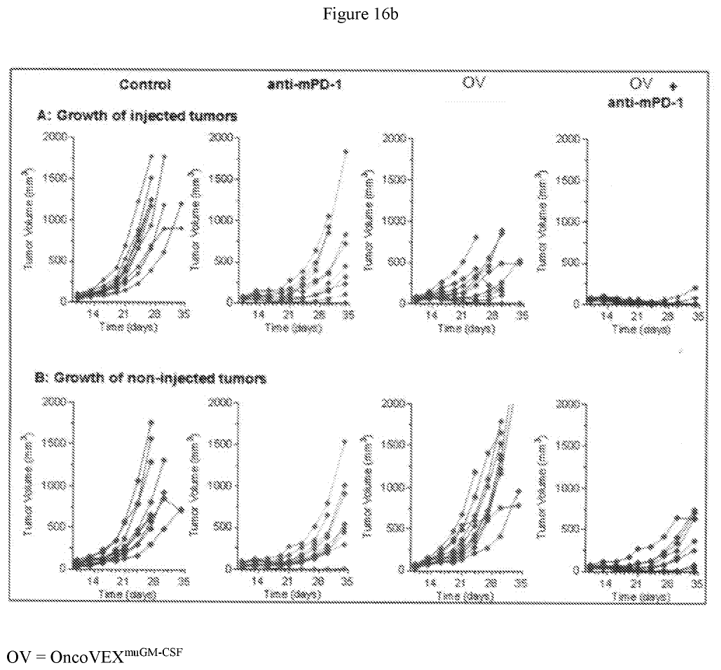

All animals survived through the experiment and showed no evidence of adverse health effects associated with treatment as evidenced by body weight (FIG. 16a) or survival, and there were no noted adverse clinical signs identified on daily health monitoring examinations. A slightly lower body weight in treated groups as compared to the control groups is attributed to more aggressive tumor growth in the control animals as compared to treated animals as discussed below.

Tumor growth inhibition was seen in response to monotherapy treatment with either anti-mPD-1 antibody (at both tested doses of 300 μg and 1 mg per dose) or OncoVEX^{muGM-CSF} (FIG. 16b). Table 11 summarizes the number of animals that were tumor free (regressions) at the end of the experiment on either the right (injected side) or left (non-injected side) flanks. Whereas single agent activity with either agent was limited to 10-20% complete regression in injected tumors (and no complete regressions in uninjected tumors), the combination led to 80-90% regression in injected tumors (and complete regressions in 10-20% of uninjected tumors). These data indicate that combination therapy with OncoVEX^{muGM-CSF} and anti-PD-1 led to substantially improved tumor clearance in the mouse MC-38 tumor model.

TABLE 11
Number of Animals with Complete Regressions

| | Injected | Uninjected |
|---|---|---|
| Group | Tumors | Tumors |
| Vehicle + Isotype | 0/10 | 0/10 |
| OncoVEX^{muGM-CSF} + Isotype | 2/10 | 0/10 |
| Vehicle + anti-PD-1 (1 mg) | 1/10 | 0/10 |
| Vehicle + anti-PD-1 (300 μg) | 1/10 | 0/10 |
| OncoVEX^{muGM-CSF} + anti-PD-1 (1 mg) | 8/10 | 2/10 |
| OncoVEX^{muGM-CSF} + anti-PD-1 (300 μg) | 9/10 | 1/10 |

These results demonstrate that OncoVex^{mGM-CSF}, either alone or in combination with PD-1 blockade, inhibits the growth of colorectal MC-38 tumors.

### Example 11

### Study Evaluating the Combination of OncoVEX^{muGm-CSF} with PD-L1 Inhibition in a Mouse Colon (Colorectal) Adenocarcinoma (MC-38) Tumor Model

This study was designed to evaluate the tolerability and anti-tumor activity of OncoVEX^{muGM-CSF}, PD-L1 inhibition, or the combination of OncoVEX^{muGM-CSF} and PD-L1 inhibition in a mouse MC-38 tumor model.

MC-38 tumor cells were injected subcutaneously in the right and left flanks of female C57BL/6 mice on day 0. Tumor volume (mm³) was measured using electronic calipers twice per week (Q2W). Once tumors reached an average of approximately 100 mm³, animals were randomized into 4 groups (10 mice per group) such that the average tumor volume (in both flanks) and the variability of tumor volume at the beginning of treatment administration were uniform across treatment groups. OncoVex^{mGM-CSF} (5×10⁶PFU/dose) or formulation buffer control were administered intratumorally (on the right side of the animal) every three days for three total injections, alone or in combination with intraperitoneal injection of anti-PD-L1 mAb (Clone MIH5, mouse IgG1) or a control IgG1 (mAbs were does four times total). The uninjected tumors (contralateral; on the left side of the animal) received no injection. Clinical signs, body weight changes, and survival (mice were removed from study when tumors reached 800 mm³) were measured 2-3 times weekly until study termination.

All animals survived through the experiment and showed no evidence of adverse health effects associated with treatment as evidenced by body weight, and there were no noted adverse clinical signs identified on daily health monitoring examinations.

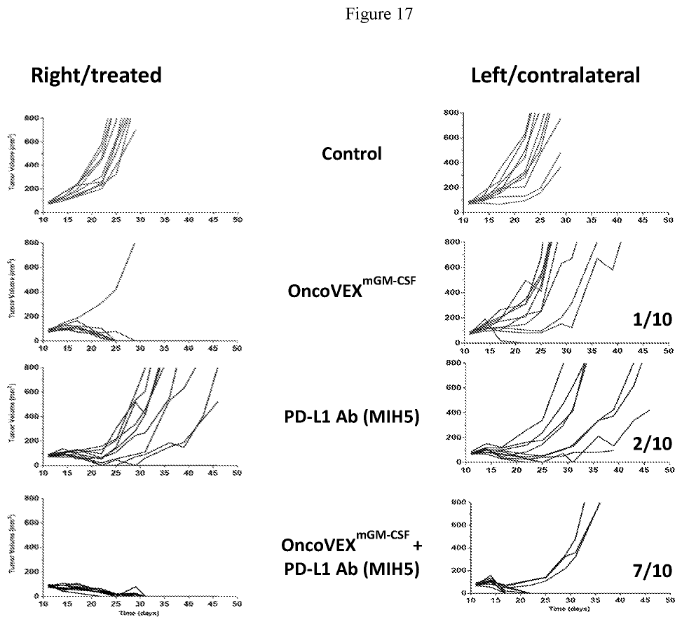

Tumor growth inhibition was seen in response to monotherapy treatment with either OncoVex^{mGM-CSF} or anti-PD-L1 mAb at both injected and contralateral tumors (FIG. 17), while the combination of OncoVex^{mGM-CSF} and anti-PD-L1 mAb regressed all injected tumors and 7 out of 10 uninjected tumors (FIG. 17),

### Example 12

### Study Evaluating the Combination of OncoVEX^{muGM-CSF} with PD-1 Inhibition in a Mouse Melanoma (B16F10) Tumor Model

This study was designed to evaluate the tolerability and anti-tumor activity of OncoVEX^{muGM-CSF}, PD-1 inhibition, or the combination of OncoVEX^{muGM-CSF} and PD-1 inhibition in a mouse melanoma B16F10 tumor model.

B16F10 tumor cells engineered to express mNectin were injected subcutaneously in the right flank of female C57BL/6 mice on day 0. Tumor volume (mm³) was measured using electronic calipers twice per week (Q2W). Once tumors reached an average of approximately 100 mm³, animals were randomized into 4 groups (10 mice per group) such that the average tumor volume and the variability of tumor volume at the beginning of treatment administration were uniform across treatment groups. OncoVex^{mGM-CSF} (5×10⁶ PFU/dose) or formulation buffer control were dosed intratumorally three times every third day, alone or in combination with intraperitoneal injection of anti-PD-1 mAb (Clone 29F1A12, mouse IgG1) or a control IgG1 (mAbs were does four times total). Clinical signs, body weight changes, and survival (mice were removed from study when tumors reached 800 mm³) were measured 2-3 times weekly until study termination.

All animals survived through the experiment and showed no evidence of adverse health effects associated with treatment as evidenced by body weight, and there were no noted adverse clinical signs identified on daily health monitoring examinations.

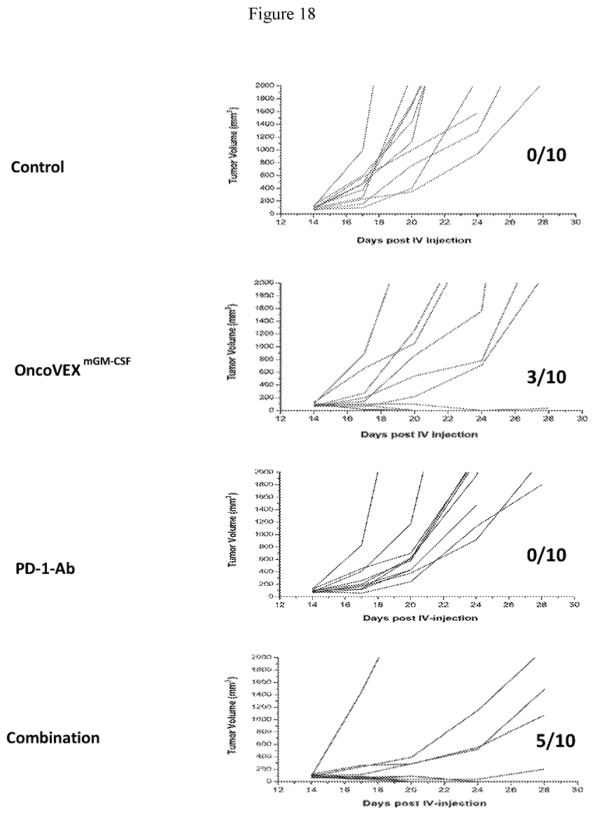

Tumor growth inhibition was seen in response to monotherapy treatment with OncoVex^{mGM-CSF} monotherapy (3 out of 10 mice showed tumor regression), while anti-PD-1 mAb monotherapy did not have any inhibitory effect on tumor growth (FIG. 18). The combination of OncoVex^{mGM-CSF} and anti-PD-1 mAb regressed 5 out of 10 injected tumors (FIG. 18), demonstrating that OncoVex^{mGM-CSF} and anti-PD-1 mAb combination has superior anti-tumor activity compared to monotherapy.

### Example 13

### Phase 1, Multi-Center, Open-Label, Dose De-Escalation Study to Evaluate the Safety and Efficacy of Talimogene Laherparepvec in Pediatric Subjects with Advanced Non-Central Nervous System Tumors that are Amenable to Direct Injection

This example is a description of a Phase 1, Multi-center, Open-label, Dose De-escalation Study to Evaluate the Safety and Efficacy of Talimogene Laherparepvec in Pediatric Subjects With Advanced Non-Central Nervous System Tumors That are Amenable to Direct Injection. See, US National Institutes of Health website (clinicaltrials.gov), under study identifier: NCT 02756845 (which is incorporated herein by reference).

The primary objective of the study is to determine the safety and tolerability of talimogene laherparepvec, as assessed by incidence of dose-limiting toxicities (DLT), in pediatric subjects with advanced non-Central Nervous System (CNS) tumors that are amenable to direct injection.

Talimogene laherparepvec will be administered to approximately 18 to 36 pediatric subjects with advanced non-CNS tumors that are amenable to direct injection. Pediatric subjects will be enrolled overall into cohorts stratified by age and baseline herpes simplex virus type-1 (HSV-1) serostatus (3 to 6 subjects/cohort). DLT will be evaluated based on 3 to 6 DLT-evaluable subjects in that cohort.

The primary outcome measure is to determine the safety and tolerability of talimogene laherparepvec, as assessed by incidence of dose-limiting toxicities (DLT), in pediatric subjects with advanced non central nervous system (CNS) tumors that are amenable to direct injection.

The secondary outcome measures are (1) to evaluate the anti-tumor activity of talimogene laherparepvec, as assessed by the overall response rate (ORR), duration of response (DOR), time to response (TTR), time to progression (TTP), progression-free survival (PFS) using modified Immune-related Response Criteria Simulating Response Evaluation Criteria in Solid Tumors (irRC-RECIST), and overall survival (OS) and (2) to evaluate the association between granulocyte macrophage colony-stimulating factor (GM-CSF) receptors/subunits in archival tumor tissue and clinical outcomes (safety endpoints and efficacy endpoints such as ORR, DOR, TTR, TTP, PFS, and OS).

Inclusion Criteria:
Subject's legally acceptable representative has provided informed consent/assent when the subject is legally too young to provide informed consent/assent and the subject has provided written assent based on local regulations and/or guidelines prior to any study-specific activities/procedures being initiated.
Male or female subjects aged 0 to <18 years at the time of informed consent/assent
Should be willing to submit local HSV-1 serostatus within 28 days prior to enrollment
Histologically or cytologically confirmed non-CNS solid tumor that recurred after standard therapy, or for which there is no standard therapy available
Presence of measurable (defined per irRC-RECIST) or non-measurable lesions as defined by modified irRC-RECIST
Subject must be a candidate for intralesional injection, defined as one or more of the following:
   at least 1 injectable lesion ≥10 mm in longest diameter
   multiple injectable lesions that in aggregate have a longest diameter of ≥10 mm
   NOTE: visceral lesions are not eligible for injection. Additionally, bone lesions are not eligible for injection unless there is a soft tissue component that is amenable to injection
Performance status:
   Karnofsky ≥70% for 12 to <18 years of age
   Lansky play scale ≥70% for children 0 to <12 years of age
Life expectancy >4 months from the date of enrollment
Adequate organ function as defined as follows:
   Hematological (without need for hematopoietic growth factor)
      absolute neutrophil count (ANC) ≥1.0×10⁹/L
      platelet count ≥75×10⁹/L
      hemoglobin ≥8 g/dL (without the need for transfusion support)
   Renal
      serum creatinine ≤1.5×upper limit of normal (ULN) for age, OR creatinine clearance ≥60 mL/min/1.73 m² for a subject with creatinine levels >1.5×ULN for age. (Note creatinine clearance need not be determined if the baseline serum creatinine is ≤1.5×ULN for age. Creatinine clearance should be determined per institutional standards).
   Hepatic
      serum bilirubin ≤1.5×ULN for age or direct bilirubin ≤ULN for age for a subject with total bilirubin level >1.5×ULN for age
      aspartate aminotransferase (AST) ≤2.5×ULN for age or ≤5×ULN for age for subject with liver metastases
      alanine aminotransferase (ALT) ≤2.5×ULN for age or ≤5×ULN for age for subject with liver metastases
   Coagulation
      international normalization ratio (INR) or prothrombin time (PT) ≤1.5×ULN for age
      partial thromboplastin time (PTT) or activated partial thromboplastin time (aPTT) ≤1.5×ULN for age
Female subject of childbearing potential should have a negative urine or serum pregnancy test within 72 hours prior to dosing. If urine test is positive or cannot be confirmed as negative, a serum pregnancy test will be required.

Exclusion Criteria
Diagnosis of leukemia, non-Hodgkin's lymphoma, Hodgkin's disease, or other hematologic malignancy
Radiotherapy to the bone marrow within 6 weeks prior to enrollment OR within 3 months prior to enrollment if prior radiotherapy to the craniospinal axis or to at least 60% of the pelvis was received; within 2 weeks prior to enrollment if local palliative radiotherapy was received
CNS tumor or clinically active brain metastases
Primary ocular or mucosal melanoma
History or evidence of giant congenital melanocytic nevi, dysplastic nevis syndrome or xeroderma pigmentosum
History of other malignancy within the past 5 years with the following exception:
   malignancy treated with curative intent and with no known active disease present and has not received chemotherapy for >5 years before enrollment and felt to be at low risk for recurrence by the treating physician
History or evidence of active autoimmune disease that requires systemic treatment (ie, with use of disease modifying agents, corticosteroids or immunosuppressive drugs). Replacement therapy (eg, thyroxine, insulin, or physiologic corticosteroid replacement therapy for adrenal or pituitary insufficiency, etc.) is not considered a form of systemic treatment.
Evidence of clinically significant immunosuppression such as the following:
   primary immunodeficiency state such as severe combined immunodeficiency disease
   concurrent opportunistic infection
   receiving systemic immunosuppressive therapy (>2 weeks prior to enrollment), including oral steroid doses (with the exception of maintenance physiologic replacement). Subjects that require intermittent use of steroids for inhalation or local steroid injection will not be excluded from the study.
Active herpetic skin lesions or prior complications of herpetic infection (eg, herpetic keratitis or encephalitis,)
Prior treatment with talimogene laherparepvec or any other oncolytic virus
Prior treatment with a tumor vaccine
Requires intermittent or chronic treatment with an antiherpetic drug (eg, acyclovir), other than intermittent topical use
Prior chemotherapy, radiotherapy, or biological cancer therapy within 28 days prior to enrollment or has not recovered to Common Terminology Criteria for Adverse Events (CTCAE) grade 1 or better from adverse event due to cancer therapy administered more than 28 days prior to enrollment
Currently receiving treatment in another investigational device or drug study, or less than 28 days since ending treatment on another investigational device or drug study(ies). Other investigational procedures while participating in this study are excluded.
Major surgery ≤28 days prior to enrollment
Expected to require other cancer therapy while on study with the exception of local palliative radiation treatment
Has acute or chronic active hepatitis B virus or hepatitis C virus infection or received treatment with nucleotide analogs such as those used in the treatment of hepatitis B virus (eg, lamivudine, adefovir, tenofovir, telbivudine, and entecavir), ribavirin, or interferon alpha within 12 weeks of initiation of study treatment.
Known or suspected human immunodeficiency virus (HIV) infection
Received live vaccine within 28 days prior to enrollment
No antiplatelet or anticoagulation medications allowed within 7 days prior to talimogene laherparepvec injection except low-dose heparin needed to maintain venous catheter patency
Female subject is pregnant or breast-feeding, or planning to become pregnant during study treatment and through 3 months after the last dose of talimogene laherparepvec
Female subject of childbearing potential who is unwilling to use acceptable method(s) of effective contraception during study treatment and through 3 months after the last dose of talimogene laherparepvec. Note: Acceptable methods of effective contraception are defined in the informed consent/assent form. Where required by local laws and regulations, additional country-specific contraception requirements may be outlined in a country-specific protocol supplement at the end of the Appendix Section of protocol.
Sexually active subjects and their partners unwilling to use a male or female latex condom to avoid potential viral transmission during sexual contact while on treatment and within 30 days after treatment with talimogene laherparepvec
Subject has known sensitivity to any of the products or components to be administered during dosing
Subject likely to not be available to complete all protocol-required study visits or procedures, and/or to comply with all required study procedures to the best of the subject and investigator's knowledge
History or evidence of any psychiatric disorder, substance abuse or any other clinically significant disorder, condition or disease (with the exception of those outlined above) that, in the opinion of the investigator or Amgen physician, if consulted, would pose a risk to subject safety or interfere with the study evaluation, procedures or completion
Subject who is unwilling to minimize exposure with his/her blood or other body fluids to individuals who are at higher risks for HSV-1 induced complications (immunosuppressed individuals, HIV-positive individuals, pregnant women, or children under the age of 1 year) during talimogene laherparepvec treatment and through 28 days after the last dose of talimogene laherparepvec

By the definitions of the inclusion and exclusion criteria, talimogene laherparepvec will be administered by intralesional injection only into injectable cutaneous, subcutaneous, nodal and other non-visceral tumors. Thus, the anticipated eligible tumor types for this study are as follows:
Bone sarcoma: Ewing and Osteosarcoma
Soft tissue sarcoma: Rhabdomyosarcoma and Non-rhabdomyosarcomatous soft tissue sarcoma
Neuroblastoma
Melanoma

The first dose of talimogene laherparepvec will be up to 4.0 mL of 10⁶ PFU/mL administered on day 1. The second injection, up to 4.0 mL of 10⁸ PFU/mL (or up to 4.0 mL of 10⁶ PFU/mL for a dose de-escalated cohort), will be administered 21 (+3) days after the initial injection (ie, no sooner than day 22 but should not be delayed more than 3 days after the 21-day time point). All subsequent injections, up to 4.0 mL of 10⁸ PFU/mL (or up to 4.0 mL of 10⁶ PFU/mL for a dose de-escalated cohort), will be administered every 14 (±3) days. The treatment cycle interval may be increased due to toxicity. The maximum volume of talimogene laherparepvec administered at any dose is 4.0 mL for any individual lesion and in any treatment. The recommended volume of talimogene laherparepvec to be injected into the tumor(s) is dependent on the size of the tumor(s) and will be determined according to the injection volume guideline in Table 12. It is recommended that each lesion should receive the maximum amount possible to inject due to tumor properties at each visit before moving on to the next lesion, using the prioritization model below and the injection volume guideline based on tumor size per

TABLE 12
Talimogene Laherparepvec Injection Volume
Guideline Based on Tumor Size

| Tumor Size (longest dimension) | Maximum Injection Volume |
|---|---|
| >5.0 cm | 4.0 mL |
| >2.5 cm to 5.0 cm | 2.0 mL |
| >1.5 cm to 2.5 cm | 1.0 mL |
| >0.5 cm to 1.5 cm | 0.5 mL |
| ≤0.5 cm | 0.1 mL |

On each treatment day, prioritization of injections is recommended as follows:
1. any new injectable tumor that appeared since the last injection
2. by tumor size, beginning with the largest tumor
3. any tumor previously seen on tumor assessment that was too small to inject that has now become large enough to inject

Subjects will be treated with talimogene laherparepvec until subjects have achieved a complete response (CR), no injectable tumors are present, confirmed progressive disease (PD) per modified irRC-RECIST, intolerance of study treatment, 24 months from the date of the first dose of talimogene laherparepvec, need for alternative anti-cancer therapy or end of study, whichever occurs first. Due to the mechanism of action, subjects may experience growth in existing tumors or the appearance of new tumors prior to maximal clinical benefit of talimogene laherparepvec. Therefore, modified irRC-RECIST will be used for response assessment.

1. A method of treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, melanoma, head and neck squamous carcinoma, hepatocellular carcinoma, gastric carcinoma, breast cancer (e.g., triple negative breast carcinoma), cutaneous T-cell lymphoma, or multiple myeloma by administering a therapeutically effective amount of an oncolytic virus.

2. The method of claim 1, wherein said oncolytic virus is a herpes simplex virus.

3. The method of claim 2, wherein said oncolytic virus is talimogene laherparepvec.

4. A method of treating B-cell lymphoma, colorectal cancer, head and neck squamous carcinoma, or breast cancer (e.g., triple negative breast carcinoma), by administering:
(i) a therapeutically effective amount of an oncolytic virus; and
(ii) a therapeutically effective amount of a checkpoint inhibitor.

5. The method according to claim 4, wherein said checkpoint inhibitor is a CTLA-4, PD-L1, or a PD-L1 blocker.

6. The method of claim 5, wherein said oncolytic virus is a herpes simplex virus.

7. The method of claim 6, wherein said herpes simplex virus is talimogene laherparepvec.

8. (canceled)

9. A pharmaceutical composition for use in a method of treating Ewing sarcoma, neuroblastoma, rhabdoid tumor, osteosarcoma, rhabdomyosarcoma, B-cell lymphoma (e.g., diffuse large B-cell lymphoma), non-small cell lung carcinoma, colorectal, head and neck squamous carcinoma, hepatocellular carcinoma, gastric carcinoma, breast cancer (e.g., triple negative breast carcinoma), cutaneous T-cell lymphoma, or multiple myeloma, wherein said pharmaceutical composition comprises an oncolytic virus.

10. The oncolytic virus of claim 9, wherein said oncolytic virus is a herpes simplex virus.

11. The oncolytic virus of claim 10, wherein said herpes simplex virus is talimogene laherparepvec.

12. (canceled)

13. A pharmaceutical composition for use in a method of treating B-cell lymphoma (e.g., diffuse large B-cell lymphoma), colorectal cancer, head and neck squamous carcinoma, or breast cancer (e.g., triple negative breast carcinoma), wherein said pharmaceutical composition comprises a therapeutically effective amount of an oncolytic virus and a checkpoint inhibitor.

14. The checkpoint inhibitor of claim 13, wherein said checkpoint inhibitor is a CTLA-4, PD-1, or PD-L1 blocker.

15. The oncolytic virus of claim 14, wherein said oncolytic virus is a herpes simplex virus.

16. The oncolytic virus of claim 15, wherein said herpes simplex virus is talimogene laherparepvec.
